(19) United States
(12) Patent Application Publication
(10) Pub. No.: US 2011/0237495 A1
(43) Pub. Date: Sep. 29, 2011

(54) FUNCTIONAL METABOLOMICS COUPLED MICROFLUIDIC CHEMOTAXIS DEVICE AND IDENTIFICATION OF NOVEL CELL MEDIATORS

(75) Inventors: Charles N. Serhan, Needham, MA (US); Rong Yang, Boston, MA (US)

(73) Assignee: The Brigham and Women's Hospital, Inc. Corporate Sponsored Research and Licensing, Boston, MA (US)

(21) Appl. No.: 12/993,603

(22) PCT Filed: May 21, 2009
(86) PCT No.: PCT/US09/44873

§ 371 Date: Jun. 1, 2011

Related U.S. Application Data
(60) Provisional application No. 61/055,022, filed on May 21, 2008.

(51) Int. Cl. A61K 38/17; A61K 31/202; A61P 19/02; A61P 19/10

(52) U.S. Cl. 514/1.4; 514/560; 514/1.1; 514/16.8; 514/16.7; 514/16.9

(57) ABSTRACT

The invention relates to the use of microfluidic chemotaxis device to identify novel active compounds of the metabolome and methods to characterize their actions on cell motility. The invention also includes the active compounds of the metabolome identified by the methods and devices disclosed herein. The results from these in vitro experiments were then correlated with in vivo physiologic responses to identify the downstream effects and therapeutic value. Thus, the invention provides novel methods for treating or preventing second organ injury resulting from ischemia-reperfusion in a patient in need thereof The invention also provides methods of treating, preventing or ameliorating connective tissue degeneration in a patient in need thereof. The invention also provides methods of treating or preventing bone loss. In addition, the invention provides method for inducing bone regeneration in patients in need thereof or preventing bone loss in patients suspected to be in need thereof.

### CROSS-REFERENCE TO RELATED APPLICATION(S)

This is a Section 371 National Stage Application of International No. PCT/US2009/044873, filed on 21 May 2009, and published as WO 2009/143363 A1 on Nov. 26, 2009, which claims priority to U.S. Provisional Patent Application No. 61/055,022, filed May 21, 2008, entitled “Functional Metabolomics Coupled Microfluidic Chemotaxis Device and Identification of Novel Cell Mediator” the entire contents of both of which are incorporated herein by reference.

### FEDERAL FUNDING

This invention was made, in part, with United States government support awarded by The National Institutes of Health grants R37-GM38765 and P50-DE016191. The Government of the United States has certain rights in this invention.

### FIELD OF THE INVENTION

The invention relates to the use of a microfluidic chemotaxis device to identify novel active compounds of the metabolome and characterize their actions on cell motility with in vivo identification of their physiologic effect. The invention also includes the active compounds of the metabolome identified by the methods and devices disclosed herein.

### BACKGROUND OF THE INVENTION

Cell motility is an important function or many cells. For some cells, motility is marked by noticeable morphological and spatial changes while, in other cells motility is more subtle. For example, cells that migrate can be identified in their migratory mode not only due to their movement across or through a matrix but, when captured in an image exhibit a polarized morphology and have easily recognized organelles such as filopodia, lamellopodia, microspikes and the like which aid in the movement of the cell toward some end point. In mammals, examples of such motile cells include fibroblasts, leukocytes and epithelial cells. Other cells exhibit more subtle forms of movement such as the growth of nerve axons toward a target or in some cases away from other axons. Such cell movements, in health are important during development such as during embryonic gastrulation, neuronal development, and tissue regeneration. In disease, migration may be an inherent part of the pathological progression such as in cell migration during cancer metastasis. As biology becomes more capable of focusing on the interaction of cells at the cellular and molecular level, it is becoming increasingly evident that motile cells respond to chemical cues which are very hard to identify due to the very small concentration of compounds in the surrounding medium.

Recently, science has advanced to allow decoding of whole genomes (genomics) and to identify their gene products, including the study of proteins and their functions (proteomics). It is becoming clear that physiologically active compounds are not limited to proteins or individual gene products but include active metabolites resulting from translational end-products and metabolites resulting from maintenance of normal cellular homeostasis as well as pathologic states of all classes of molecules including, proteins and peptides, carbohydrates, lipids, fatty acids, including saturated and polyunsaturated fatty acids and, in some cases, nucleic acids. Thus, a new field of science termed “metabolomics” is evolving which attempts to provide a systematic study of unique chemical fingerprints that specific cellular process leave behind. Therefore, the metabolome represents the collection of all metabolites in a biological organism, which are products of gene expression, whether the direct end product or metabolites of those end products. Further, while in many cases the exact mechanism of action by which autocrine agents exert their cellular effects has not been worked out, the ability to use cell motility as a model represents an important tool in dissecting the metabolome. For example, current research indicates that ligands such as epidermal growth factor (EGF) may stimulate cell motility in fibroblasts by binding to the EGF receptor (EGFR) possibly by affecting the cytoskeleton. As noted above, the difficulty with dissecting the metabolome is that when exerting paracrine or autocrine effects, the active agents are present in extremely small quantities. Thus, the ability to identify active agents or ligands and/or their receptors and identify the effect they exert has not been possible. Further, with the advent of genomics and proteomics, the identification of many receptor molecules that have no known ligand has become evident. These receptors are called “orphan receptors” and provide just a small indication of the possible effector molecules or ligands that comprise part of the metabolome that have yet to be discovered.

Other examples include cellular processes that were once thought to be merely passive that are slowly being recognized as active process entering and resolving disease states. For example, mounting evidence indicates that the resolution of acute inflammation is a highly coordinated and biochemically active process that was once thought to be a passive event¹ Neutrophils migrate into tissues to participate in host defense, and then these tissues return to their homeostatic functions². Consequently, tissue level resolution programs are actively initiated and the number of PMN are reduced¹. Hence it is possible that excessive inflammatory responses and their progression to chronic inflammation might result from a local failure to resolve¹ the inflammatory processes. The processes by which acute inflammation is resolved toward tissue homeostasis are of considerable interest since many widely occurring human diseases are associated with chronic inflammation. These diseases include, but are not limited to, arthritis³ and periodontal disease⁴ as well as diseases that are recognized relatively recently to have aberrant inflammation as a component of the disease including asthma, cardiovascular disease and Alzheimer's disease⁵⁻⁷.

The process of inflammation resolution is controlled in part by formation of newly described endogenous chemical mediators termed “autacoids” that stimulate pro-resolving mechanisms, previously discussed (see refs.^{1, 8}). These novel pro-resolving mediators are derived from essential fatty acids that include arachidonic acid (AA, C20:4), eicosapentaenoic acid (EPA, C20:5) and docosahexaenoic acid (DHA, C22:6)⁸. These local mediators are biosynthesized during spontaneous resolution and when administered in vivo, they stimulate multi-cellular tissue level responses geared to bring tissues back to cellular and molecular homeostasis in a process termed “catabasis”⁹.

Lipoxins (LXs), biosynthesized from AA, were the first mediators recognized to carry both potent anti-inflammatory and pro-resolving actions⁹⁻¹¹. LXs reduce PMN infiltration. They also stimulate non-phlogistic recruitment of monocytes and enhance macrophage take-up of apoptotic PMN¹² as well as clearance of microbial particles⁹. Of note, LXs are not immunosuppressive because they stimulate anti-microbial activities of mucosal epithelial cells¹³ and attenuate inflammation-induced pain by direct actions on neural tissues¹. The impact of omega (ω)-3 fatty acids (EPA and DHA) has been evaluated in numerous clinical studies¹⁴⁻¹⁸. Of interest are the findings from the GISSI (Gruppo Italiano per lo Studio della Sopravvivenza nell'Infarto miocardico) studies that revealed the significant benefits of ω-3 fatty acid supplementation in cardiovascular disease. These reports and many others emphasize the potential benefits of dietary supplementation with ω-3 fatty acids. For example, the i.v. administration of ω-3 fatty acids leads to clinical improvements in patients with rheumatoid arthritis. In healthy subjects, ω-3 fatty acids reduce LPS-induced fever as well as inflammatory responses¹⁷. In addition, soy nuts, enriched with ω-3 fatty acids, improve systolic blood pressure and low-density lipoprotein cholesterol levels in hypertensive postmenopausal women¹⁸. It has further been found that ω-3 fatty acids reduce risk of type 1 diabetes, in at-risk children.¹⁹

DHA is widely appreciated for its neurotrophic and neuroprotection roles that require esterification into phospholipids^{20,21}. DHA tissue levels also appear to be critical since in diseases such as cystic fibrosis the DHA stores appear to be depleted²². The ω-3 fatty acids are precursors to potent stereoselective families of mediators, namely resolvins and protectins that are biosynthesized in resolving exudates¹. Recently, the original structural elucidation of resolvin D1 (RvD 1) was confirmed by total organic synthesis, and its complete stereochemistry was established as 7S,8R,17S-trihydroxy-4Z,9E,11E,13Z,15E,19Z-docosahexaenoic acid^{23,24}. Thus, the relationship between circulating levels of EPA and DHA in plasma is of interest and a focus of the inventors' research. The values of ω-3 fatty acids have been measured in many studies. In humans, they show a wide range of values; in healthy subjects, for example, EPA ranges from ˜0.6-2.8% of total fatty acids and DHA from ˜1.3-5.0%²⁵⁻²⁹. This wide range of values for humans appears to reflect diet²⁹. Since ω-3 fatty acids are now recognized as biosynthetic precursors to potent mediators their circulating blood levels may be relevant in inflammation and its resolution. For example, Table 1 provides for a direct comparison of varying values which may be correlated with culture and diet.

TABLE 1

| Reference | Subjects | EPA and DHA levels | Note |
|---|---|---|---|
| Gong et al., | Control group | EPA 0.49 ± 0.04 (%) | % of Plasma fatty |
| (1992)21 | (n = 91) | DHA 1.34 ± 0.06 (%) | acids |
| Newcomer et al., | Control group | EPA 0.61 ± 0.36 (%) | % of Total fatty acids |
| (2001)22 | (n = 156, Men) | DHA 4.17 ± 1.26 (%) | in RBC |
| Albert et al., | Control group | EPA 1.84 ± 0.53 (%) | % of Total fatty acids |
| (2002)23 | (n = 184) | DHA 2.38 ± 0.78 (%) | in blood |
| Kew et al., | Placebo control, Baseline | EPA 0.7 ± 0.3 (%) | % of Total fatty acids |
| (2004)24 | (n = 8-12) | DHA 2.8 ± 1.2(%) | in neutrophils |
| Wakai et al., | Healthy subjects | Women | % of Total fatty acids |
| (2005)25 | (n = 1257: Japanese, | EPA 2.47(%) | in serum |
| | Women: 626, Men: 631) | DHA 4.93(%) | |
| | | Men | |
| | | EPA 2.82(%) | |
| | | DHA 5.07(%) | |

Thus, the ability to identify and study the composition of and effects of putative active agents of the metabolome will provide previously unrecognized ability to influence cellular physiology both in health and disease. This ability is, in part, predicated on being able to observe and identify the effects of micro, nano or even pico molar concentrations of putative agents on individual cells of varying types.

### SUMMARY OF THE INVENTION

The invention relates to the use of microfluidic chemotaxis device to identify novel active compounds of the metabolome and methods to characterize their actions on cell motility. The invention also includes the active compounds of the metabolome identified by the methods and devices disclosed herein. The results from these in vitro experiments were then correlated with in vivo physiologic responses to identify the downstream effects and therapeutic value. Thus, the invention provides novel methods for treating or preventing second organ injury resulting from ischemia-reperfusion in a patient in need thereof. The invention also provides methods of treating, preventing or ameliorating connective tissue degeneration in a patient in need thereof. The invention also provides methods of treating or preventing bone loss. In addition, the invention provides method for inducing bone regeneration in patients in need thereof or preventing bone loss in patients suspected to be in need thereof.

Therefore, in one exemplary embodiment, the invention comprises a device for identifying mediators of cellular motility comprising: a microfluidic chemotaxis device, wherein the microfluidic chemotaxis device requires a volume of up to 100 μl. In some exemplary embodiments, the microfluidic chemotaxis device is adapted to require a volume of up to 104 In other exemplary embodiments, the invention further includes one or more gradient generators. In still other exemplary embodiments, the invention further comprises a chemotaxis assay chamber. In some exemplary embodiments, the invention further includes one or more microscale valves. In still other exemplary embodiments, the invention includes an ability to capture and image individual cells. In various exemplary embodiments, the microfluidic chemotaxis device is coated with a capture molecule to select a specific cell type to be captured. In some exemplary embodiments, the capture molecule is a cellular adhesion molecule, an antibody, a partial antibody, a ligand or a receptor. In various embodiments, the cellular adhesion molecule is an NCAM, an ICAM-1, a VCAM-1, a PECAM, an L1-CAM, CRL1, a myelin-associated glycoprotein adhesion molecule, an integrin a cadherin or a selectin. In various exemplary embodiments, the individual cells captured are fibroblasts, leukocytes epithelial cells, neurons, muscle cells, hair cells, sperm, or unicellular organisms having cilia or flagella such as those in the phyla protozoa or coelenterata. In some exemplary embodiments, the chemotaxis assay chamber and/or the one or more gradient chambers are coated with a polyethylene glycol solution. In various exemplary embodiments, the one or more microvalves are pneumatically controlled from the outside of the chamber. In some exemplary embodiments, the one or more gradient generator contains a chemokine. In some exemplary embodiments, the chemokine is a CC chemokine, a CXC chemokine, a C chemokine, or a CX₃C chemokine. In various exemplary embodiments a putative motility mediator is added to the chemotaxis assay chamber. In these exemplary embodiments, the putative motility mediatory is a metabolome product.

In still another exemplary embodiment, the invention comprises a method of inhibiting cell motility comprising inhibiting cell motility with an effective amount of an active metabolome mediator compound. In various exemplary embodiments according to the invention, the mediator compound is a protein, a peptide, a carbohydrate, a lipid, a fatty acid, including saturated and polyunsaturated fatty acids or a nucleic acid. In some exemplary embodiments the metabolome mediator compound is a lipid mediatory. In various exemplary embodiments, the resolvin is a D series resolvin or an E series resolvin. In some embodiments, the resolvin or protectin compound is administered orally, rectally, topically, intravenously, intraperitoneally, as an inhalant, as a mist, as a tablet, capsule or tincture. In these exemplary embodiments, the motile cells are fibroblasts, leukocytes epithelial cells, neurons, muscle cells, hair cells, sperm, or unicellular organisms having cilia or flagella such as those in the phyla protozoa or coelenterata.

In still another exemplary embodiment, the invention comprises a method of treating or preventing second organ injury resulting from ischemia-reperfusion comprising administering to a patient suffering or having the potential to suffer from ischemia-reperfusion an effective amount of an inflammation resolving compound or a protectin compound. In various exemplary embodiments, the inflammation resolving compound is a D series resolvin or an E series resolvin. In various exemplary embodiments the diseases treated or ameliorated by the instant invention include, but are note limited to, transplant surgery, bypass surgery, septic shock, and explant surgery. In some exemplary embodiments, the inflammation resolving compound is administered orally, rectally, topically, intravenously, intraperitoneally, as an inhalant, as a mist, as a tablet, capsule or tincture. In still other exemplary embodiments, the inflammation resolving compound is applied as a cream, lotion, emollient, gel, ointment or liquid.

In yet other exemplary embodiments, the invention includes a system for identifying the effectiveness of mediators of cell motility comprising, providing a microfluidic chemotaxis device capable of visualizing a single motile cell; capturing an individual motile cell; establishing a chemotaxis gradient in the microfluidic chemotaxis device; inserting in the microfluidic chemotaxis device a potential motility mediating compound; identifying the effect of motility of the potential motility mediating compound. In these exemplary embodiments, the microfluidic chemotaxis device further comprises a one or more gradient chambers. In still other exemplary embodiments, the microfluidic chemotaxis device further comprises a chemotaxis assay chamber. In various other exemplary embodiments, the motile cell is captured from a physiological fluid by a capture molecule. In various exemplary embodiments, the capture molecule is a cellular adhesion molecule, an antibody, a partial antibody, a ligand or a receptor. In some exemplary embodiments, the cell adhesion molecule is an NCAM, an ICAM-1, a VCAM-1, a PECAM, an L1-CAM, CRL1, a myelin-associated glycoprotein adhesion molecule, an integrin a cadherin or a selectin. In various exemplary embodiments, the physiological fluid is blood, saliva, synovial fluid, cerebrospinal fluid or lymph. In various other exemplary embodiments, the potential mediating compound is derived from a physiologic medium. In some exemplary embodiments, the physiologic medium is blood, plasma, synovial fluid, cerebrospinal fluid or lymph, saliva, an exudate, a transudate, a juice, a concentrate. In various exemplary embodiments, the potential mediating compound is a metabolome product. In various exemplary embodiments, the metabolome product is a protein, a peptide, a carbohydrate, a lipid, a fatty acid, including saturated and polyunsaturated fatty acids or a nucleic acid.

In still another exemplary embodiment of the invention, the invention includes a compound useful for treating or preventing connective tissue degeneration in a patient in need thereof, comprising a non-metabolizable analog of a resolvin or a protectin. In various exemplary embodiments the compound is a p-fluoro analogue of the resolvin D family or the resovlin E family of lipid mediators. In various embodiments, the compound is administered orally, rectally, topically, intravenously, intraperitoneally, as an inhalant, as a mist, as a tablet, capsule or tincture. In still other exemplary embodiments, the compound is contained within a matrix and the matrix is implanted at the site of the connective tissue degeneration. In various exemplary embodiments, the matrix is a hydrogel.

In yet another exemplary embodiment, the invention includes a method of treating connective tissue degeneration comprising, administering to a patient in need thereof, an effective amount of a resolvin or a protectin. In various exemplary embodiments, the resolvin or protectin is a non-metabolizable analog. In some exemplary embodiments, the compound is a p-fluoro analogue of the resolvin D family or the resovlin E family of lipid mediators. In various exemplary embodiments, the connective tissue degeneration is a result of arthritis, degenerative joint disease, diabetes, gout, lyme disease, perthes' disease, osteoarthritis, mechanical injury, alkaptonuria, or hemochromatosis. In some exemplary embodiments, the resolvin or protectin analog is contained within a matrix and is implanted in a joint.

In yet another exemplary embodiment, the invention includes a method of identifying mediators of cell motility comprising, using a using a microfluidic chemotaxis device having a volume of less than about 20 μl; introducing a chemokine into a gradient generator of the microfluidic chemotaxis device; introducing a putative chemotaxis mediator into a chemotaxis assay chamber of the microfluidic chemotaxis device; introducing a motile cell into the chemotaxis assay chamber; and observing the motility of the cell. In various exemplary embodiments, a chemokine gradient is produced between the chemotaxis assay chamber and one or more gradient generators. According to one exemplary embodiment, the microfluidic chemotaxis device allows for the visualization of a single motile or potentially motile cell in response to the chemokine gradient. In yet another exemplary embodiment, the invention provides for the visualizing the effects of a potential chemotaxis mediator on the single motile cell. According to some aspects of this exemplary embodiment, motile cell or potentially motile cell is captured from physiologic medium by attraction to a capture molecule. In some exemplary embodiments, the physiologic medium is water, blood, saliva, synovial fluid, cerebrospinal fluid, water or lymph. In other aspects of this exemplary embodiment, the capture molecule is a cellular adhesion molecule, an antibody, a partial antibody, a ligand or a receptor. In various exemplary embodiments, the ligand is a cellular adhesion molecule. In some exemplary embodiments, the cellular adhesion molecule is an NCAM, an ICAM-1, a VCAM-1, a PECAM, an L1-CAM, CHL1, a myelin-associated glycoprotein adhesion molecule, an integrin a cadherin or a selectin. In some preferred embodiments the cell adhesion molecule is P-selectin. In various exemplary embodiments, the chemokine is selected from CC chemokines, CXC chemokines, C chemokines or CX₃C chemokines. In some exemplary embodiments, the chemokine is a CXC chemokine. In some embodiments, the chemokine is IL-8. In various exemplary embodiments, the putative mediator is derived from a physiologic medium. In various exemplary embodiments, the motile cell or potentially motile cell is a leukocyte a fibroblast, a epithelial cell, a neuron, a muscle cell, a hair cell, a sperm, or a unicellular organism having cilia or flagella such as those in the phyla protozoa or coelenterata. In some exemplary embodiments, the leukocyte is a PMN.

In various exemplary embodiments, the putative mediator is a protein, a peptide, a carbohydrate, lipids, fatty acids, including saturated and polyunsaturated fatty acids or a nucleic acid. In some exemplary embodiments, the fatty acid is a lipid mediator. In some exemplary embodiments the lipid mediator is a member of the resolvin D family or the resolvin E family of lipid mediators. In other exemplary embodiments, the putative mediators is an analogue of a naturally occurring mediator. In some aspects the analogue is poorly metabolized and has a longer half-life than the native mediator. In various embodiments, the mediator analogue is a synthetic analogue. In some exemplary embodiments, the motile cell is associated with inflammation and the still other aspects the chemotaxis mediator is associated with inflammation. In various exemplary embodiments, the motile cell is a cell associated with inflammation and chemotaxis mediator is associated with resolving inflammation. In these and other exemplary embodiments, observing the motility of the cell in the chemotaxis assay chamber allows for the identification of members and relationships of active compounds of the metabolome.

It should be understood that, according to the methods of the invention described above, the invention provides a device and methods for identifying relationships of the components of the metabolome. Further, according to various exemplary embodiments according to the invention, the identification of the actions of the members of the metabolome provide compounds and methods to mediate cell motility and modulate the effects of disease in which cell motility has a pathologic effect. Such diseases are exemplified, but not limited to, those demonstrating inflammation and metastasis

It should be appreciated that the invention can be administered in any suitable way. For example, in various exemplary embodiments, the invention can be administered topically, orally, parenterally, transdermally or rectally.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be apparent from the description, as set forth hereinafter.

### BRIEF DESCRIPTION OF THE FIGURES

Various exemplary embodiments of the compositions and methods according to the invention will be described in detail, with reference to the following figures.

FIG. 1 is a cartoon identifying some of the lipid mediators discussed herein and their contribution to inducing and resolving acute inflammation that accompanies tissue injury and disease.

FIG. 2 is a cartoon showing LMs, including resolvins (Rv) and protectins (PC) and their aspirin triggered (AT) epimers, actively regulate neutrophils in vivo.

FIG. 3 is a cartoon illustrating the biosynthetic pathway of the resolvin D series compounds originating with the DHA precursor.

FIGS. 4A-D are mass spectra identifying deuterium-labeled omega-3 fatty acids (d₅-EPA (FIG. 4A) and d₅-DHA (FIG. 4B) and the methyl parinate control (FIG. 4C) and identifying their appearance in inflammatory exudates (FIG. 4D). FIG. 4A) Mass spectrum of d₅-EPA methyl ester and SIM chromatogram of d₅-EPA in mouse exudate. The ω-3 ion ((CH)₃CH₂(CH)₂CD₂CD₃=113) and α ion 180 were selected as diagnostic ions. The retention time was 11.7 min. FIG. 4B) Mass spectrum of d₅-DHA methyl ester and SIM chromatogram of d₅-DHA in mouse exudate. The ω-3 ion 113 and α ion 160 were selected as diagnostic ions. The retention time was 13.9 min. FIG. 4D) is a plot of the time course of d₅-EPA and d₅-DHA in peritoneal exudates. Upper panel: Results shown as % of i.v. injection; closed squares: d₅-EPA; closed circles: d₅-DHA. Results represent the mean±SEM from n=3. *, significantly different from 24 h, p<0.05. Middle panel: Time course of PMN infiltration. Closed squares: Zymosan A treatment; closed circles: Zymosan A+d₅-EPA and d₅-DHA. Results represent the mean±SEM from n=3. Lower panel: Time course of total protein exudation. Total exudate extracellular protein levels were determined by the Lowry method and expressed as total amount in each³⁹. Results represent the mean±SEM from n=7˜20. *, significantly different from 0 h, p<0.05. ‡, significantly different from 4 h, p<0.05.

FIG. 5 is a graph illustrating the rapid exudate appearance of ¹⁴C-DHA from the circulation. Each exudate was mixed with scintillation fluid and radioactivity was counted with a scintillation counter. Extracellular protein levels were determined by the Bradford method and expressed as the total amount in each sample. Three different groups of animals and experiments were performed.

FIGS. 6A and B show the results of FACS analysis of peritoneal leukocyte composition. FIG. 6A zymosan A-induced acute inflammation; 6B) zymosan A-induced acute inflammation+d₅-EPA and d₅-DHA. Lavage cells were stained with PE-conjugated anti-mouse Ly-6G and PerCP-Cy5.5-conjugated anti-mouse CD11b. Ly-6G is primarily expressed on PMN while CD11b is expressed on both PMN and monocytes.

FIGS. 7A-D are data showing that RvD1 protects from second organ lung injury following ischemia/reperfusion. FIG. 7A (top panel) illustrates the time course of the experiment. FIG. 7B is a photomicrograph showing a hematoxylin/eosin-stained lung from ischemia/reperfusion second organ injury (control). Magnification: ×60; ×20 for inset. FIG. 7C) shows hematoxylin/eosin-stained lung from 1 μg RvD1 i.v. injection to ischemia/reperfusion second organ injury model followed by i.v. administration of RvD1. Magnification: ×60. FIG. 7D) is a graph quantifying the results of the experiments illustrated in FIGS. 7B and C and showing that that RvD1, but not DHA or RvE1 (data not shown) protects lung from ischemia/reperfusion injury. I/R=ischemia/reperfusion.

FIGS. 8A-C are micrographs showing the newly disclosed microfluidic chemotaxis device for studying PMN chemotaxis from whole blood according to one exemplary embodiment of the invention. FIG. 8A) is an overview of the microvolume chemotaxis device adapted for isolating individual neutrophils from whole blood and studying successive exposure of the cells to pre-formed chemokine gradients and lipid mediators. FIG. 8B) shows that rapid switching between chemotaxis conditions is achieved through the use of microvolume chemotaxis valves integrated on a chip, and pneumatically controlled from the outside. FIG. 8C) illustrates that a single drop of blood from, for example, a finger prick can be used in the device.

FIGS. 9A-C show data illustrating that RvD1 inhibits neutrophil chemotaxis. FIG. 9A) are photomicrographs showing that neutrophils having polarized morphology, during exposure to IL-8 gradient (−1 min), quickly become rounded (+1 min) and are never able to recover their polarized morphology (+5 min) after exposure to the lipid mediator RvD1. FIG. 9B) is a graph showing the average displacement of control neutrophil (migration) in an IL-8 gradient. This migration is immediately and effectively blocked after exposure to RvD1. Results represent the mean±SEM, n=12. FIG. 9C is a control showing neutrophil migration on P-selectin surface without IL-8. No significant chemotaxis is observed for neutrophils toward a P-selectin surface in the absence of IL-8. At a few minutes after establishing a chemoattractant IL-8 gradient, neutrophils display sustained migration in the direction of the gradient. Results represent the mean±SEM, n=12, for each value.

FIG. 10 is a graph providing a direct comparison of DHA and RvD1 in an IL-8 gradient with PMN isolated from a drop of whole blood. This figure illustrates that DHA, precursor of RvD1, failed to stop neutrophil chemotaxis while chemotaxis immediately ceased following addition of RvD1. Closed circle, RvD1; open triangle, DHA. Results represent the mean±SEM, n=12, for each.

FIG. 11 is a graph showing that resolvins and related analogs are protective in vivo. The graph illustrates the percent reduction of MPO found in lungs after administration of the indicated compounds in the ischemia-reperfusion experiments described in Example 12 and 13. Structures of the compounds used in this experiment are shown above their effect on leukocyte population is reported in the graph plotting reduction in MPO. Test compounds [1 μg of RvD1 carboxymethyl ester (RvD1-Me), 17-(R/S)-methyl-RvD1 carboxymethyl ester (17-(R/S)-methyl-RvD1-Me), and 19-p-fluorophenoxy-RvE1 methyl ester (19-p-fluorophenoxy-RvE1-Me)] in vehicle were administered intravenously. Results indicate mean±SEM (n=3-5, control; n=12). RvD1-Me, 22.9 ±6.8, n=4; 17-(R/S)-methyl-RvD1-Me, 29.3 ±6.4, n=3; 19-p-fluorophenoxy-RvE1-Me, 19.7 ±7.7, n=3.*, significantly different from values obtained with vehicle, p<0.02. †, significantly different from values obtained with DHA, p<0.01.

FIGS. 12A-12C clinical photographs showing the soft tissue and bone response in rabbit to RvE1 topical monotherapy vs. controls. FIG. 12A shows a healthy rabbit periodontium. The left panel shows the soft tissue, the right panel is the same preparation defleshed to show the underlying bone. FIG. 12B shows the effect of induced periodontitis with vehicle (placebo) treatment. FIG. 12C shows that when periodontitis was induced (as in FIG. 12B) RvE1 treatment stimulated regeneration of lost periodontal structures e.g., both soft tissue and bone.

FIG. 13 is a graph illustrating that RvE1 induces regeneration of bone. Of note, animals with induced periodontitis (light blue bars) that were treated with RvE1 (yellow bars) showed significant bone regeneration (approximately 80%) when compared with healthy controls (dark blue bars).

FIGS. 14A-14C illustrates the effect of resolving treatment on the regeneration of the bone and connective tissue of the rabbit periodontium. FIG. 14A micrograph showing the undecalcified ground section of the regenerated rabbit periodontium illustrated in FIG. 12C. FIG. 14B is a phase contrast microscopic image. FIG. 14C is the same preparation using a polarized light microscope. These images illustrate deposition of new cementum (NC) and new periodontal ligament (PL), connective tissue (CT) and bone (B).

FIG. 15 shows that resolvins inhibit the differentiation of monocytes into osteoclasts. This effect is even more pronounced than for PRP (platelet rich plasma) a recognized inhibitor of osteoclast differentiation. Preparations of 5, 10, 20% PRP are compared with RvE1 were quantified in experiments in which peripheral blood monocytes were induced to differentiate into osteoclasts with RANKL treatment. Treatment showed marked inhibition of osteoclast differentiation and activity induced by PRP and RvE1 (P<0.05 for all treatments).

### DETAILED DESCRIPTION OF THE EXEMPLARY EMBODIMENTS

The invention relates to the use of microfluidic chemotaxis device to identify novel active compounds of the metabolome and methods to characterize their actions on cell motility. The invention also includes the active compounds of the metabolome identified by the methods and devices disclosed herein. The results from these in vitro experiments were then correlated with in vivo physiologic responses to identify the downstream effects and therapeutic value. Thus, the invention provides novel methods for treating or preventing second organ injury resulting from ischemia-reperfusion in a patient in need thereof The invention also provides methods of treating, preventing or ameliorating connective tissue degeneration in a patient in need thereof The invention also provides methods of treating or preventing bone loss. In addition, the invention provides method for inducing bone regeneration in patients in need thereof or preventing bone loss in patients suspected to be in need thereof

A microfluidic chemotaxis device is disclosed capable of capturing a single cell from a sample as small as 5 μl. The microfluidic chemotaxis device comprises a chemotaxis chamber, a gradient chamber and microscale valves. The microfluidic chemotaxis device provides for real-time imaging of motile cells. In some particularly preferred embodiments, the cells are inflammatory cells and, in particular PMN. The invention also provides compounds and methods to treat degenerative tissue disease as well as second organ injury resulting from ischemia-reperfusion. The compounds and methods include administration of a synthetic resolvin or protectin compound that is not easily metabolized and therefore, has a longer half-life and activity.

As a model, the inventors have identified that a well-integrated inflammatory response and its natural resolution are essential to cellular homeostasis and health¹⁵. Therefore, the inventors have developed methods and devices directed to achieve a complete understanding of the molecular events that govern termination of acute inflammation. The inventors have recognized that the compounds of these molecular events comprise members of the inflammation metabolome and that the process of activating inflammation and resolving inflammation are normal processes that, in health, act to protect the body from trauma and disease. However, in certain disease states, the process of inflammation is not resolved and results in or potentiates the diseased state itself. During the course of their research, the inventors realized that by identifying the active members of the metabolome they could be used to mediate and modulate the disease state, whether specifically part of the inflammation response or other. In inflammation, the response is predicated on cell motility and migration of cells into the affected area. Identification of active metabolome compounds useful in modulating the response provides valuable tools for mediating the effects of diseases and disease states in which cell motility and migration plays a part. Cells that exhibit motility include but are not limited to fibroblasts, leukocytes and epithelial cells neurons, muscle cells, hair cells, sperm, or unicellular organisms having cilia or flagella such as those in the phyla protozoa or coelenterata. Identification of active compounds that affect such cells both in health and disease provides tools for research and for clinical use.

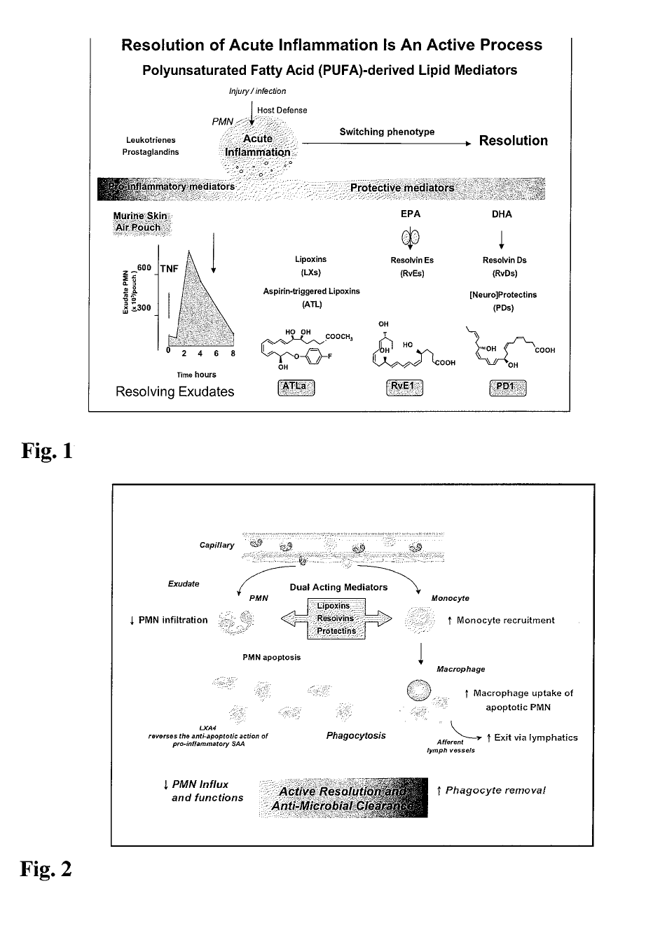

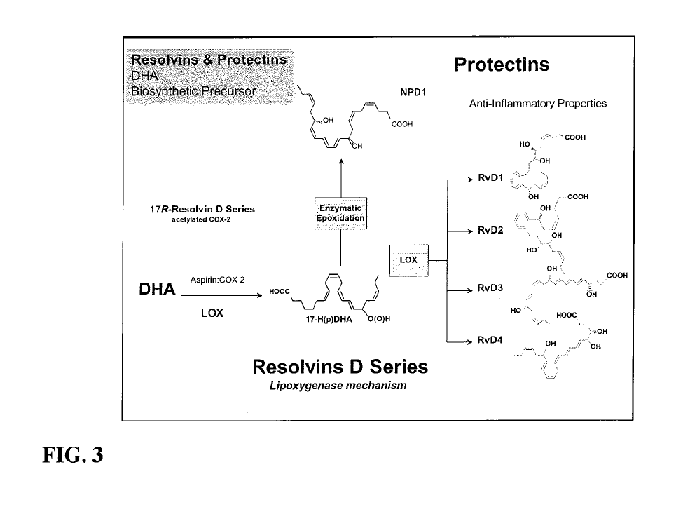

For example, recent studies by the inventors have identified endogenous biochemical pathways from inflammatory exudates taken during the resolution phase of inflammation. FIG. 1 illustrates that resolution of the acute phase is an active process and that new families of locally acting mediators (autocoids) are actively generated from the essential fatty acids eicosapentaenoic acid (EPA) and docosahexaenoic acid (DHA). These fatty acids are widely known as the omega-3 (ω-3) or fish oil lipids that are held to play beneficial roles in many diseases¹⁶, particularly those associated with inflammation. The omega-3 essential fatty acids have proven to be the precursors of newly identified chemical mediator families termed “resolvins” and “protectins”^{11,17,18} because, in animals, specific members of these families control the duration and magnitude of inflammation³ and are pro-resolving⁵. These actions are illustrated in FIG. 2. Together these endogenous mediators comprise a new genus of anti-inflammatories that are pro-resolving agonists rather than inhibitors. Hence, mapping of these resolution circuits, mediators and the target signaling pathways of these potent agonists of inflammation resolution provide new tools for modulating the molecular basis of many inflammatory diseases and mediating their pathology.

The inventors recent advances on the biosynthesis and actions of these novel anti-inflammatory lipid mediators, with a focus on the stereochemical basis of the potent actions of resolvin E1¹⁹ and protectin D1²⁰ and have been reviewed⁵. These previously unappreciated families of lipid-derived mediators were originally isolated from experimental murine models of acute inflammation recovered during natural self-limited resolution. Since they have proven anti-inflammatory and pro-resolving and have shown protective properties in in vitro and in vivo models of disease. Due to their potent and promising effects, it is important to establish the direct actions of these resolvins and protectins, particularly RvD1, RvE1 and their analogs, with human neutrophils. These investigations will further help to assign their complete stereochemistry.

### Definitions

The following abbreviations are used throughout the text.
AA: arachidonic acid [5Z,8Z,11Z,14Z-eicosa-5,8,11,14-tetraenoic acid;
CAM: cellular adhesion molecule
DHA: docosahexaenoic acid, [4Z,7Z,10Z,13Z,16Z,19Z]-docosa-4,7,10,13,16,19-hexaenoic acid;
EFA: essential fatty acid;
EPA: eicosapentaenoic acid, [5Z,8Z,11Z,14Z,17Z-icosa-5,8,11,14,17-pentaenoic acid;
IL-8: interleukin-8 a chemokine produced by macrophages
I/R: ischemia reperfusion injury;
LX: lipoxin;
MPO: myeloperoxidase;
RvE1: resolvin E1, 5S,12R,18R-trihydroxy-6Z,8E,10E,14Z,16E-eicosapentaenoic acid;
RvD1: resolvin D1, 7S,8R,17S-trihydroxy-4Z,9E,11E,13Z,15E,19Z-docosahexaenoic acid;
PD1/NPD 1: protectin D1/neuroprotectin D1, 10R,17S-dihydroxy-docosa-4Z,7Z,11E,13E,15Z,19Z-hexaenoic acid;
PMN: polymorphonuclear neutrophils;
PDMS: poly(dimethylsiloxane).

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific ten is used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

“Subject” and “patient” are used interchangeably and mean mammals and non-mammals. “Mammals” means any member of the class mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

As used herein, “administering” or “administration” includes any means for introducing an active compound of the metabolome into the body, preferably into the systemic circulation. Examples include but are not limited to oral; buccal, sublingual, pulmonary, transdermal, transmucosal, as well as subcutaneous, intraperitoneal, intravenous, and intramuscular injection.

For purposes of the present invention, “treating” or “treatment” describes the management and care of a patient for the purpose of combating the disease, condition, or disorder. The terms embrace both preventative, i.e., prophylactic, and palliative treatment. Treating includes the administration of a compound of present invention to prevent the onset of the symptoms or complications, alleviating the symptoms or complications, or eliminating the disease, condition, or disorder.

As used herein the word “autocoid” refers to a chemical substance produced by one type of cell that affects the function of different types of cells in the same region, thus functioning as a local hormone or messenger. As used herein, paracrine is a form of cell signaling in which the target cell is close by is of a different cell type. Autocrine refers to cell signaling in which the target cell is a local cell of the same cell type.

As used herein the term “catabasis” refers to the stage of decline or resolution of a disease. As used herein the term “metabolome” refers to the complete set of small-molecule metabolites (such as metabolic intermediates, hormones and other signaling molecules, and secondary metabolites) to be found within a biological sample, such as a single organism. The metabolome varies with the physiologic states or developmental state of the cell or organism.

As used herein the tenn “capture molecule” means any molecule capable of capturing another molecule. Capture molecules can be antibodies or fragments of antibodies that have binding domains such as Fab's or partial Fab's. Capture molecules can be “ligands” or “receptors” either partial or complete and whether or not they are associated with other molecules or compounds. As used herein the term “ligand” refers a molecule that binds to another molecule. Generally, a ligand may be soluble and its binding partner is referred to as a “receptor”. Unless otherwise defined, receptors are generally regarded as being associated with particular cells. Classically, receptors transduce a signal conferred by a ligand with the binding of the ligand to the receptor resulting in a signal to the cell and a physiologic response. Receptors may be on the cell surface on within the cells. As a rule, peptide or protein hormones are ligands that bind to receptors at the cell surface while steroid hormones are ligands that pass through the cell membrane and bind to receptors in the cell nucleus. As discussed below, receptors can become soluble (as in P-selectin, below) and act as ligand. Thus, the terminology of “receptor” and “ligand” may become interchangeable. Generally, a receptor is thought to be larger than its corresponding ligand.

As used herein, the term “cell adhesion molecule” (CAM) refers to any of the families of proteins that are located on the cell surface (membrane receptors) and are involved with the binding with other cells of the extracellular matrix. E.g., proteins involved in cell adhesion. CAMs are typically transmembrane receptors are have n intracellular domain that interacts with the cytoskeleton, a transmembrane domain and an extracellular domain that interacts with other CAMs of the same kind with other CAMS of a different kind or with the extracellular matrix. Such families of CAMs have been described an include, but are not limited to, immunoglobulin superfamily cams (IgSF CAMs) including neural cell adhesion molecules (NCAMs), intercellular cell adhesion molecules (ICAM-1), vascular cell adhesion molecule (VCAM-1), platelet-endothelial cell adhesion molecule (PECAM-1), L1 protein cellular adhesion molecule (L1-CAM), CHL1 adhesion molecule and the myelin-associated glycoprotein adhesion molecule (MAG, Siglec-4). Other families of CAM include the integrins, the cadherins and the selectins. As used herein, P-selectin refers to a cell adhesion molecule found in granules in endothelial cells lining blood vessels and plays a role in the recruitment of leukocytes. P-selectin can be released by endothelial cell upon injury to result in the local recruitment of leukocytes to the site of injury.

As used herein the term “chemokine” refers to a family of small cytokines or proteins that are secreted by cells (ligands). Cytokines bind to cytokine receptors which are cell surface glycoproteins and are members of the seven-transmembrane g-protein-coupled receptor family. Chemokines have the ability to induce directed chemotaxis in nearby responsive cells. The chemokines are classified according to shared structural characteristics such as, size, the presence of cytokine residues in conserved locations. Currently the chemokines are categorized into four main groups: (1) the CC chemokines which have to adjacent cysteines near their amino terminus; (2) the CXC chemokines which are characterized by having two cysteines near their amino terminus separated by another amino acid; (3) the C chemokines, characterized by the having only two cysteines, one N-terminal and one downstream; and (4) the CX₃C chemokines characterized by having three amino acids between the N-terminal cysteines. As used herein “chemotaxis” refers to a response of motile cells or organisms in which the direction of movement is affected by the gradient of a diffusible substance.

As used herein, motile cell means any cell that is capable of movement either in a healthy state or a diseased state. Such cell may be migratory and move distances, such as leukocytes, fibroblasts or endothelial cells or they may grow toward an endpoint such as the growth of a neuronal axon toward a complementary dendrite. Further, some motile cells may only exhibit motility during a portion of their life such cells include, but are not limited to cells of the blastula during embryonic development. In addition, some cells may only be motile in disease states. Examples include cancer cells during the process of metastasis. Other cells may have motile components such as the cilia of hair cells and epithelia of the respiratory tract and flagella of sperm. In addition, other motile cells may include single celled organisms such as protozoans and coelenterates. It should be appreciated that the motile cells of present invention are not limited to those of mammals but any cell having or capable of having motility whether single-celled or from a multi-cellular organism.

As used herein, “physiologic medium” refers to any medium having a physiologic basis. Such mediums may be found in vivo or ex vivo. For example, the physiologic medium may be a liquid such as blood or lymph or it may result from a cellular extract or a medium used to support cells such as, for example, water, Luria broth (lb) or the like.

As used herein the term “native mediator” means a mediator of cell chemotaxis or homeostasis that is found in nature. An analogue may be found in nature or it may be synthetically made. Some analogues are poorly metabolized by the native mediator's metabolic pathway and, therefore, have a longer half-life in the body than does the native mediator.

As used herein “inflammatory disease” can mean any disease which produces inflammation or for which inflammation is a symptom. As used herein inflammation-reperfusion refers to cellular damage after reperfusion of previously viable ischemic tissues. In some instances reperfusion after ischemia results in greater tissue damage than prior to the reperfusion. As used herein, “second organ injury” refers to remote organ injury occurring down stream from an occluded vessel and in response to the release of activated neutrophils at the site of occlusion.

As used herein a peptide is a compound of two to about 100 amino acids linked by peptide bonds. As used herein a protein is a compound of more than about 100 amino acids linked by peptide bonds.

### The Invention

The invention relates to the use of microfluidic chemotaxis device to identify novel active compounds of the metabolome and methods to characterize their actions. The results from these in vitro experiments was then correlated with in vivo physiologic responses to identify the downstream effects and therapeutic value. Thus, the invention also includes the active compounds of the metabolome identified by the methods and devices disclosed herein. A microfluidic chemotaxis device is disclosed capable of capturing a single cell from a sample as small as 5 μl. The microfluidic chemotaxis device comprises a chemotaxis chamber, a gradient chamber and microscale valves. The microfluidic chemotaxis device provides for real-time imaging of motile cells. In some particularly preferred embodiments, the cells are inflammatory cells and, in particular PMN. The invention also provides compounds and methods to treat degenerative tissue disease as well as second organ injury resulting from ischemia-reperfusion. The compounds and methods include administration of a synthetic resolvin or protectin compound that is not easily metabolized and therefore, has a longer half-life and activity.

Therefore, in one exemplary embodiment, the invention comprises a device for identifying mediators of cellular motility comprising: a microfluidic chemotaxis device, wherein the microfluidic chemotaxis device requires a volume of up to 100 μl. In some exemplary embodiments, the microfluidic chemotaxis device is adapted to require a volume of up to 10 μl. In other exemplary embodiments, the invention further includes one or more gradient generators. In still other exemplary embodiments, the invention further comprises a chemotaxis assay chamber. In some exemplary embodiments, the invention further includes one or more microscale valves. In still other exemplary embodiments, the invention includes an ability to capture and image individual cells. In various exemplary embodiments, the microfluidic chemotaxis device is coated with a capture molecule to select a specific cell type to be captured. In some exemplary embodiments, the capture molecule is a cellular adhesion molecule, an antibody, a partial antibody, a ligand or a receptor. In various embodiments, the cellular adhesion molecule is an NCAM, an ICAM-1, a VCAM-1, a PECAM, an L1-CAM, CHL1, a myelin-associated glycoprotein adhesion molecule, an integrin a cadherin or a selectin. In various exemplary embodiments, the individual cells captured are fibroblasts, leukocytes epithelial cells, neurons, muscle cells, hair cells, sperm, or unicellular organisms having cilia or flagella such as those in the phyla protozoa or coelenterata. In some exemplary embodiments, the chemotaxis assay chamber and/or the one or more gradient chambers are coated with a polyethylene glycol solution. In various exemplary embodiments, the one or more microvalves are pneumatically controlled from the outside of the chamber. In some exemplary embodiments, the one or more gradient generator contains a chemokine. In some exemplary embodiments, the chemokine is a CC chemokine, a CXC chemokine, a C chemokine, or a CX₃C chemokine. In various exemplary embodiments a putative motility mediator is added to the chemotaxis assay chamber. In these exemplary embodiments, the putative motility mediatory is a metabolome product.

In still another exemplary embodiment, the invention comprises a method of inhibiting cell motility comprising inhibiting cell motility with an effective amount of an active metabolome mediator compound. In various exemplary embodiments according to the invention, the mediator compound is a protein, a peptide, a carbohydrate, a lipid, a fatty acid, including saturated and polyunsaturated fatty acids or a nucleic acid. In some exemplary embodiments the metabolome mediator compound is a lipid mediatory. In various exemplary embodiments, the resolvin is a D series resolvin or an E series resolvin. In some embodiments, the resolvin or protectin compound is administered orally, rectally, topically, intravenously, intraperitoneally, as an inhalant, as a mist, as a tablet, capsule or tincture. In these exemplary embodiments, the motile cells are fibroblasts, leukocytes epithelial cells, neurons, muscle cells, hair cells, sperm, or unicellular organisms having cilia or flagella such as those in the phyla protozoa or coelenterata.

In still another exemplary embodiment, the invention comprises a method of treating or preventing second organ injury resulting from ischemia-reperfusion comprising administering to a patient suffering or having the potential to suffer from ischemia-reperfusion an effective amount of an inflammation resolving compound or a protectin compound. In various exemplary embodiments, the inflammation resolving compound is a D series resolvin or an E series resolvin. In some exemplary embodiments, the inflammation resolving compound is administered orally, rectally, topically, intravenously, intraperitoneally, as an inhalant, as a mist, as a tablet, capsule or tincture. In still other exemplary embodiments, the inflammation resolving compound is applied as a cream, lotion, emollient, gel, ointment or liquid.

In yet other exemplary embodiments, the invention includes a system for identifying the effectiveness of mediators of cell motility comprising, providing a microfluidic chemotaxis device capable of visualizing a single motile cell; capturing an individual motile cell; establishing a chemotaxis gradient in the microfluidic chemotaxis device; inserting in the microfluidic chemotaxis device a potential motility mediating compound; identifying the effect of motility of the potential motility mediating compound. In these exemplary embodiments, the microfluidic chemotaxis device further comprises a one or more gradient chambers. In still other exemplary embodiments, the microfluidic chemotaxis device further comprises a chemotaxis assay chamber. In various other exemplary embodiments, the motile cell is captured from a physiological fluid by a capture molecule. In various exemplary embodiments, the capture molecule is a cellular adhesion molecule, an antibody, a partial antibody, a ligand or a receptor. In some exemplary embodiments, the cell adhesion molecule is an NCAM, an ICAM-1, a VCAM-1, a PECAM, an L1-CAM, CHL1, a myelin-associated glycoprotein adhesion molecule, an integrin a cadherin or a selectin. In various exemplary embodiments, the physiological fluid is blood, saliva, synovial fluid, cerebrospinal fluid or lymph. In various other exemplary embodiments, the potential mediating compound is derived from a physiologic medium. In some exemplary embodiments, the physiologic medium is blood, plasma, synovial fluid, cerebrospinal fluid or lymph, saliva, an exudate, a transudate, a juice, a concentrate. In various exemplary embodiments, the potential mediating compound is a metabolome product. In various exemplary embodiments, the metabolome product is a protein, a peptide, a carbohydrate, a lipid, a fatty acid, including saturated and polyunsaturated fatty acids or a nucleic acid.

In still another exemplary embodiment of the invention, the invention includes a compound useful for treating or preventing connective tissue degeneration in a patient in need thereof, comprising a non-metabolizable analog of a resolvin or a protectin. In various exemplary embodiments the compound is a p-fluoro analogue of the resolvin D family or the resovlin E family of lipid mediators. In various embodiments, the compound is administered orally, rectally, topically, intravenously, intraperitoneally, as an inhalant, as a mist, as a tablet, capsule or tincture. In still other exemplary embodiments, the compound is contained within a matrix and the matrix is implanted at the site of the connective tissue degeneration. In various exemplary embodiments, the matrix is a hydrogel or a miniature osmotic pump.

In yet another exemplary embodiment, the invention includes a method of treating connective tissue degeneration comprising, administering to a patient in need thereof, an effective amount of a resolvin or a protectin. In various exemplary embodiments, the resolvin or protectin is a non-metabolizable analog. In some exemplary embodiments, the compound is a p-fluoro analogue of the resolvin D family or the resovlin E family of lipid mediators. In various exemplary embodiments, the connective tissue degeneration is a result of arthritis, degenerative joint disease, diabetes, gout, lyme disease, perthes' disease, mechanical injury, alkaptonuria, or hemochromatosis. In some exemplary embodiments, the resolvin or protectin analog is contained within a matrix and is implanted in a joint.

In still other exemplary embodiments, the invention provides methods of preventing, treating or ameliorating bone loss comprising, administering to a patient in need thereof, an effective amount of a resolvin or a protectin. In some exemplary embodiments, the bone loss results from osteoporosis, arthritis or periodontal disease. In various exemplary embodiments the In some exemplary embodiments, the compound is a p-fluoro analogue of the resolvin D family or the resolvin E family of lipid mediators. In various embodiments the compounds are administered prophylactically to a patient at risk of suffering bone loss. In other embodiments the compounds are administered therapeutically to a patient suffering bone loss.

In yet another exemplary embodiment, the invention includes a method of identifying mediators of cell motility comprising, using a using a microfluidic chemotaxis device having a volume of less than about 20 μl; introducing a chemokine into a gradient generator of the microfluidic chemotaxis device; introducing a putative chemotaxis mediator into a chemotaxis assay chamber of the microfluidic chemotaxis device; introducing a motile cell into the chemotaxis assay chamber; and observing the motility of the cell. In various exemplary embodiments, a chemokine gradient is produced between the chemotaxis assay chamber and one or more gradient generators. According to one exemplary embodiment, the microfluidic chemotaxis device allows for the visualization of a single motile or potentially motile cell in response to the chemokine gradient. In yet another exemplary embodiment, the invention provides for the visualizing the effects of a potential chemotaxis mediator on the single motile cell. According to some aspects of this exemplary embodiment, motile cell or potentially motile cell is captured from physiologic medium by attraction to a capture molecule. In some exemplary embodiments, the physiologic medium is water, blood, saliva, synovial fluid, cerebrospinal fluid, water or lymph. In other aspects of this exemplary embodiment, the capture molecule is a cellular adhesion molecule, an antibody, a partial antibody, a ligand or a receptor. In various exemplary embodiments, the ligand is a cellular adhesion molecule. In some exemplary embodiments, the cellular adhesion molecule is an NCAM, an ICAM-1, a VCAM-1, a PECAM, an L1-CAM, CRL1, a myelin-associated glycoprotein adhesion molecule, an integrin a cadherin or a selectin. In some preferred embodiments the cell adhesion molecule is P-selectin. In various exemplary embodiments, the chemokine is selected from CC chemokines, CXC chemokines, C chemokines or CX₃C chemokines. In some exemplary embodiments, the chemokine is a CXC chemokine. In some embodiments, the chemokine is IL-8. In various exemplary embodiments, the putative mediator is derived from a physiologic medium. In various exemplary embodiments, the motile cell or potentially motile cell is a leukocyte a fibroblast, a epithelial cell, a neuron, a muscle cell, a hair cell, a spenn, or a unicellular organism having cilia or flagella such as those in the phyla protozoa or coelenterata. In some exemplary embodiments, the leukocyte is a PMN.

In various exemplary embodiments, the putative mediator is a protein, a peptide, a carbohydrate, lipids, fatty acids, including saturated and polyunsaturated fatty acids or a nucleic acid. In some exemplary embodiments, the fatty acid is a lipid mediator. In some exemplary embodiments the lipid mediator is a member of the resolvin D family or the resolvin E family of lipid mediators. In other exemplary embodiments, the putative mediators is an analogue of a naturally occurring mediator. In some aspects the analogue is poorly metabolized and has a longer half-life than the native mediator. In various embodiments, the mediator analogue is a synthetic analogue. In some exemplary embodiments, the motile cell is associated with inflammation and the still other aspects the chemotaxis mediator is associated with inflammation. In various exemplary embodiments, the motile cell is a cell associated with inflammation and chemotaxis mediator is associated with resolving inflammation. In these and other exemplary embodiments, observing the motility of the cell in the chemotaxis assay chamber allows for the identification of members and relationships of active compounds of the metabolome.

It should be understood that, according to the methods of the invention described above, the invention provides a device and methods for identifying relationships of the components of the metabolome. Further, according to various exemplary embodiments according to the invention, the identification of the actions of the members of the metabolome provide compounds and methods to mediate cell motility and modulate the effects of disease in which cell motility has a pathologic effect. Such diseases are exemplified, but not limited to, those demonstrating inflammation and metastasis.

During the course of spontaneous resolution of acute inflammation, ω-3 fatty acids are precursors for the biosynthesis of anti-inflammatory and pro-resolving lipid mediators^{1, 8}. These newly recognized families of mediators, termed “resolvins” and “protectins”, were identified in vivo and serve as autocoids in molecular circuits that actively promote resolution of local inflammation³⁹. As disclosed herein, evidence for these new mechanisms is presented which indicate that unesterifed omega-3 fatty acids also known as “free” fatty acids rapidly appear within the inflammatory exudates moving from circulation into the site of inflammation paralleling the movements of both albumin and trafficking leukocytes. Also, single cell analyses of human PMN using a newly engineered microfluidic chemotaxis device, described below, provide direct evidence that resolvin D1 at nanomolar concentrations, and not its precursor DHA at equimolar levels, stops PMN chemotactic responses to gradients of the cheniokine IL-8. Resolvins are active on target cells in the immediate milieu and are then inactivated by carbon position specific metabolism^{30, 44}. Further, the results disclosed herein show that, to enhance their actions, analogs of both RvD1 and RvE1 that delay their local inactivation, proved to protect organs in vivo from ischemia-reperfusion injury. Thus, omega-3 levels in circulating blood are rapidly made available to sites of inflammation in vivo for their local conversion in resolving exudates to potent bioactive mediators i.e., resolvins and protectins, that act directly on target cells to stimulate anti-inflammation and resolution; they are subject to local inactivation to permit tissues to return to homeostasis.

After ingestion, EPA and DHA are distributed throughout the human body. Results from cross-study analysis indicate that DHA is predominantly distributed in retina, sperm, cerebral cortex, spleen and red blood cells, whereas EPA is found in quite low levels in muscle, liver, spleen and red blood cells⁴⁵. For example, DHA is esterified in phospholipids of microglial cells in culture and on activation of these cells DHA is released from the phospholipids for enzymatic processing^{46,47}. DHA is the precursor to two separate families of mediators that are structurally distinct, namely D series resolvins and protectins. These protectins possess potent biological actions and a conjugated triene double structure as distinguishing features²³. EPA is the precursor for E series resolvins that show potent actions in several complex disease models, including IBD, periodontal diseases and asthma (reviewed in ref.⁴⁸). However, the timing and state of these unesterified or free ω-3 fatty acids and their arrival in these organs and/or whether specific pools of substrate are mobilized for processing during inflammation-resolution was of interest in the present studies.

The level of total fatty acids in human blood is ˜343 mg/100 ml plasma⁴⁹. Based on this value, between 60 to 500 mg each of the total EPA and DHA may exist in human blood as basal levels. These ω-3 fatty acids are derived from the diet, from supplements, and de novo biosynthesis. The contribution of de novo ω-3 fatty acids to the total amount in healthy human subjects, however, is quite low. The proportion of a-linolenic acid converted to EPA is likely on the order of 0.20 to 8.0%, and the extent of conversion of α-linolenic acid to DHA is 0.05 to 4.0%^{50,51}. Thus, humans need to ingest ω-3 fatty acids via diet and/or supplementation. Currently, the FDA states that the dietary intake of EPA and DHA should not exceed 3 g/day⁵² since supplementation on the order of a gram was found to reduce EPA and DHA biosynthesis⁵⁰.

Although DHA and EPA are widely believed to possess anti-inflammatory properties themselves, the specific mechanisms responsible for these actions are still being identified. The omega-3 fatty acids are generally thought to replace the sn-2 position in phospholipid stores that is usually the positional site of esterified n-6 fatty acids such as arachidonic acid⁵³. Hence, upon activation, cells release arachidonic acid from the sn-2 position in phospholipids via cytosolic phospholipase A2 and it is converted to eicosanoids. Among the potent bioactive eicosanoids produced, by leukocytes, for example, the prostaglandins and leukotrienes are broadly considered pro-inflammatory⁵⁴. Thus, the sn-2 position phospholipid substituted n-3 omega fatty acids (DHA, EPA) can compete for these enzymatic reactions blocking the utilization of arachidonate and subsequent production of the eicosanoid inflammatory and pro-thrombotic mediators. To address these points in the present investigations, the inventors determined the kinetics of appearance of EPA and DHA, precursors of resolvins and protectins, at local sites of inflammation in vivo. Given that both EPA and DHA move along with peripheral blood flow and are distributed in the circulation, the inventors assessed whether circulating EPA and DHA would appear at sites of inflammation in forming exudates coincident with albumin and leukocyte trafficking. The presence of albumin at sites of inflammation, by definition, determines whether the inflammatory site is considered an inflammatory exudate or transudate⁴¹. The main protein component in the inflammatory exudates generated in zymosan initiated peritonitis is, indeed, serum albumin demonstrated by 2D-gel electrophoresis and proteomics³⁹.

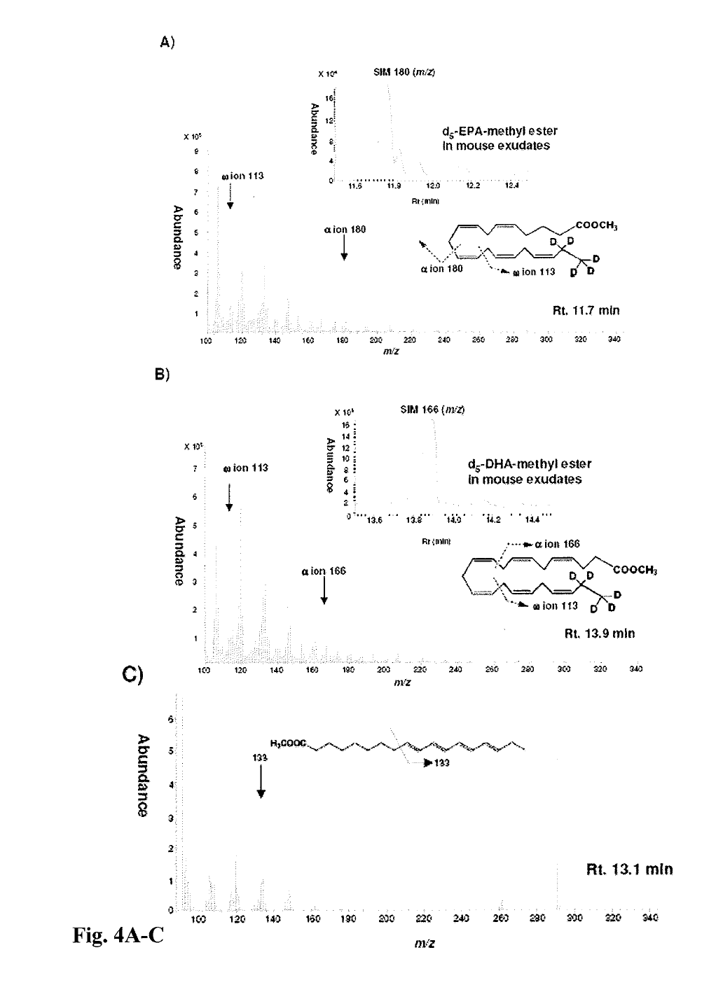

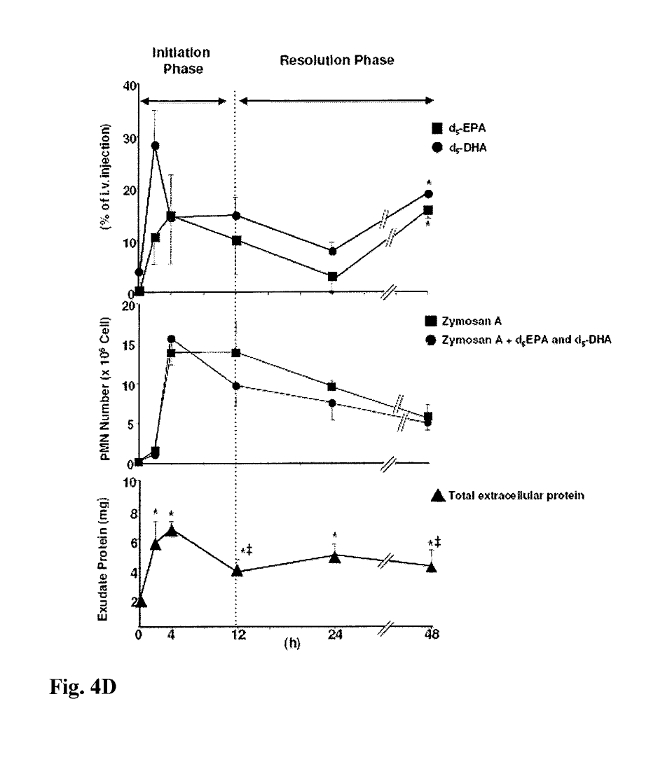

Albumin is a well appreciated carrier protein of unesterified fatty acids⁵⁵. In the experiments disclosed herein, the inventors monitored both deuterium labeled d₅-EPA and d₅-DHA levels as well as protein levels in exudates and found that they appear coincident in the foaming exudates as shown in FIG. 4D. FIG. 4D shows that both d₅-EPA and d₅-DHA (top panel) were identified in exudates within 1 h of initiation of inflammation and maintained levels up to 48 h. At 48 h, both d₅-EPA and d₅-DHA levels were significantly greater within the exudates than their levels at 24 h. Since in human plasma, the half-life of EPA is 67 h and that of DHA is 20 h⁵¹, the present results suggest that circulating ω-3 fatty acids can be made available at sites of acute inflammation directly from the circulation. Hence the present results indicate that circulating plasma DHA and EPA are directly utilized by developing inflammatory exudates and do not require specialized mobilization from complex lipids to mediate inflammation and effect its timely resolution.

Microfluidic Chemotaxis Device

The inventors have developed a new microfluidic chemotaxis device to rapidly isolate individual cells from microvolumes of physiologic media. While most easily used as a fluid, such media may be prepared as an extract in order to load the cells into the chamber. Generally, such media includes, blood, saliva, lymph, semen, exudate, transudate, extract, culture media such as Luria broth or water including but not limited to wastewater, effluent from sewerage etc. as described previously, one problem previously presented with prior chemotaxis chambers was that, in order to study the desired cell type, it was necessary to first, purify the desired cell from the physiologic media and second prior chemotaxis chambers are large requiring large volumes of buffer, media and or cells to operate the chambers. Thus, the problem incurred with such devices is that 1) it is difficult to observe a single cell; 2) due to the large volumes required by the chamber, the effect of physiologic concentrations of active compounds is difficult and 3; in order to provide the volume of desired cells necessary, the extraction and purification of such cells is required. this purification process results in damage and decrease in the viability of the desired cells. for example, in Boyden chambers⁵⁷, one cannot directly observe the cells, the information obtained is indirect, and the system requires a large number of cells. In the Dunn and Zigmond chambers^{58,59}, it is not possible to swiftly switch between cell incubation/exposure conditions as with the present microfluidic chemotaxis devices.

The present invention allows for individual cells to be captured directly from their milieu and provides for the use of a very small volume of medium, between 2-10 μl and allows for the recording of real time changes in the morphology of the captured cells. Further, due to the small volume of the chamber, it is possible to use micro valves to control a chemokine gradient precisely allowing for rapid changes in the gradient and washout of the chamber without otherwise harming or stressing the cell. Those of skill in the art will appreciate that such characteristics are not possible with large volume chambers.

For example, in some embodiments, human neutrophils were isolated directly from circulating whole blood via capture on a P-selectin coated surface. This approach allowed the inventors to asses the direct actions of both putative active compounds and further, compare their effects to precursors. In one exemplary embodiment the actions of precursor compounds, such as, for example, DHA, and EPA and their metabolites resolvins of the D and E family were tested on individual PMNs for chemotactic responses. As discussed above, previous methods require careful, time consuming isolation of neutrophils from whole blood prior to in vitro analyses. This process involves several steps of centrifugation and red blood cell lysis that usually takes several hours⁵⁶. Thus, the isolated neutrophils do not provide the best model for study. The P-selectin based capture of neutrophils requires only a single drop of whole blood (4-5 μl) and the nutrophil isolation much more closes mirrors migration of in vivo scenarios where neutrophils roll on the endothelial surfaces, stick to the endothelium in regions of higher selectin expression, and respond via chemotaxis in the gradient of chemokine e.g. IL-8 and migrate into tissues (see ref.⁴¹).

The small amount of blood used in the microfluidic chemotaxis device is an advantage for perfoiming these analyses with human PMN because it circumvents the need for phlebotomy and the risks associated. A key feature of this system is the ability to record real-time changes in morphology of PMN upon exposure to chemokines, DHA and lipid mediators such as resolvin D1, as well as to track migration through switches. Neutrophils were isolated and available for these chemotaxis studies on average within less than 5 minutes. This short time interval is ideal for assessing the activation and /or inhibition status of neutrophils from the blood of healthy donors as well as patients. The fast gradient switches in the device also allowed visual assessment and recording of the earliest events after exposure to resolvin Dl or native DHA as well as precise measurement of these change in migration direction and velocity. Currently, other chemotaxis systems are not available that allow this level of precision or time resolution.

For instance, in Boyden chambers⁵⁷, one cannot directly observe the cells, the information obtained is indirect, and the system requires a large number of cells. In the Dunn and Zigmond chambers^{58,59}, it is not possible to swiftly switch between cell incubation/exposure conditions as with the present microfluidic chemotaxis devices. The engineered microstructured valves allowed different protocols in separate sections of the device. The surface of the gradient generator network channels was coated with a PEG derivative to reduce surface adsorption of the test compounds e.g. DHA or RvD1. The surface of the main channel was modified with P-selectin specifically for capturing neutrophils directly from whole blood. Thus, another advantage of this system is that the same neutrophils served as positive controls for migration in a chemoattractant gradient as well as, probed with either RvD1 or its precursor native DHA. The preservation of chemoattractant gradient before and after the switch is important for avoiding neutrophil responses to sudden changes of chemoattractant gradient. Of interest, sudden decrements in the concentration of chemokines has the potential to stop neutrophil migration for 3-5 minutes^{36,60}. Thus, the present direct assessment of DHA with PMN indicates that DHA itself is not a potent bioactive stop signal for PMN but rather requires conversion to RvD1.

Ischemia-Reperfusion

Ischemia-reperfusion is an event of significant clinical importance, as it commonly occurs during surgical procedures, particularly involving extremities, causing local and remote organ injury as well as increasing time costs associated with prolonged post-operative recovery⁴². When vessels are surgically clamped or occluded, the stasis of blood leads to local ischemia and the neutrophils become activated which upon release of the occlusion gives rise to second organ injury by the activated leukocytes that reach, for example, the lung³⁵. The inventors investigated the direct actions of resolvins and related stable analogues, comparing the actions of RvD1, its 17-(R/S)-methyl analogue, RvE1, and its 19-p-fluorophenoxy analogue in ischemia-reperfusion second organ injury. It was found that RvD1 and its analogue as well as the stable analog of RvE1 showed potent anti-leukocyte actions reducing infiltration in lung tissues. RvE1 itself was not able to protect the lung presumably because of local inactivation. Of interest, RvE1 itself is both anti-inflammatory and pro-resolving in several inflammatory disease models⁴⁸. Recently, RvE1 was also shown to have potent actions in preventing joint damage and cartilage destruction in collagen-induced rodent arthritis⁶¹. Both RvD1 and RvE1 undergo site specific metabolic inactivation^{30,44}. Thus, as disclosed herein, the resolvin D1 and RvE1 analogs that display potent organ protective actions may provide new approaches to reduce organ damage characterized by excessive PMN infiltration. PMN are now appreciated to play an important role in the pathogenesis of many chronic inflammatory diseases such as arthritis (Tanaka et al., 2006).

Identification of Metabolome Actives

One goal of the inventors present research is to identify active compounds of the metabolome. One model used has provided novel lipid mediators using mediator lipidomics LC-MS-MS-based informatics in tandem with microfluidic chemotaxis device that directly act on leukocyte functions critical for the resolution of acute inflammation¹. Invasion by microbes and mild tissue injury stimulate inflammation that is normally “self-limited”. Until recently, the resolution phase was histologically defined as series of essential cellular processes that lead from acute inflammation to tissue homeostasis, widely believed to be a passive process. Inflammation associated diseases are a significant public health burden and give rise to local tissue destruction that increases in frequency and severity with age². In the inventors laboratory³⁻⁵ and now in others⁶⁻⁹, biochemical and in vivo evidence has emerged indicating that resolution is an active process with the identification of novel specialized lipid-derived mediators (LM) the inventors termed resolvins and protectins that regulate key events in acute inflammation and tissue injury (FIG. 1).

As the results disclosed herein show, it is evident that the majority of LM within resolving exudates remain to be identified in the resolution metabolome. As discussed, the inventors have undertaken the systematic elucidation of resolution phase lipid mediators using “mediator-lipidomics” for which the inventors have been developing, in vivo murine disease models in conjunction with single cell monitoring of leukocyte responses using microfluidic chemotaxis device.¹⁰⁻¹³ In other work, the inventors found that novel LM, including resolvins (Rv), protectins (PD), and their aspirin-triggered (AT) epimers, actively regulate neutrophils (PMN) and macrophages in vivo¹⁴ as shown schematically in FIG. 2. Since PMN are key in the initial release of pro-inflammatory products that damage organs and tissues,^{2,15} this present work focuses on the identification of novel lipid derived mediators that counter-regulate PMN motility and stimulate pro-resolving actions of macrophages that actively clear and remodel tissues to terminate the inflammatory response.

In the present investigation, the inventors had specific aims: (1) establish a model for the identification and characterization of compounds of the metabolome; (2) identify the accuracy of a model using the structures and actions of RvD, PD, their intermediates and further metabolites in resolution of inflammation with PMN in a microfluidic chemotaxis device. The model disclosed herein uses mediator-lipidomics for unbiased profiling of novel pathways in exudates and human PMN; and (3) identify novel lipid derived signals that evoke pro-resolving functions with leukocytes. The inventors recognized that because only small transient amounts (i.e. picogram to nanogram) of local acting mediators are produced in vivo during the resolution of inflammation, a microfluidic chemotaxis device using volumes as low as 1 microliter (1 μL) are essential in being able to identify compounds of the metabolome and in particular, the LM that are the responsible chemokine mediators in the resolution metabolome of inflammation.

Compounds Useful in the Invention

The present invention, in one embodiment, is drawn to uses described throughout the specification with isolated therapeutic agents generated from the interaction between a dietary omega-3 polyunsaturated fatty acid (PUFA) such as eicosapentaenoic acid (EPA) or docosahexaenoic acid (DHA), an oxygenase, such as cyclooxygenase-II (COX-2), and an analgesic, such as aspirin (ASA). Surprisingly, careful and challenging isolation of previously unknown and unappreciated compounds are generated from exudates by the combination of components in an appropriate environment to provide di- and tri-hydroxy EPA and DHA derivatives having unique structural and physiological properties. The present invention therefore provides for many new useful therapeutic di- and tri-hydroxy derivatives of EPA or DHA that diminish, prevent, or eliminate the disorders, conditions and/or diseases described herein.

Resolvins, such as resolvin El (RvE1; 5S,12R,18R-trihydroxyeicosapentaenoic acid) are novel anti-inflammatory lipid mediators derived from omega-3 fatty acid eicosapentaenoic acid (EPA).

The di- and tri-hydroxy EPA and DHA therapeutic agents of the invention useful to treat the disorders, conditions and/or diseases include, for example:

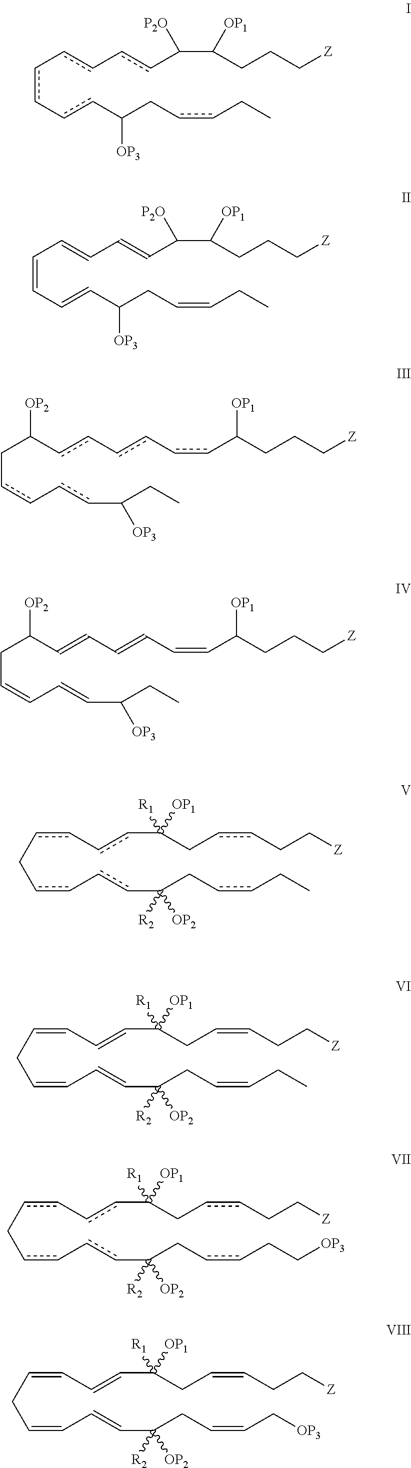

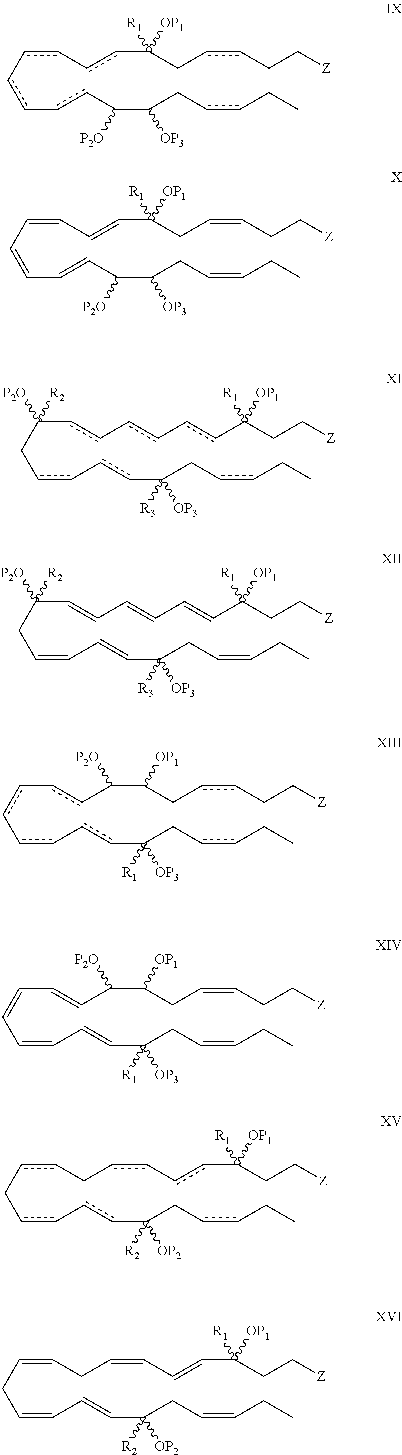

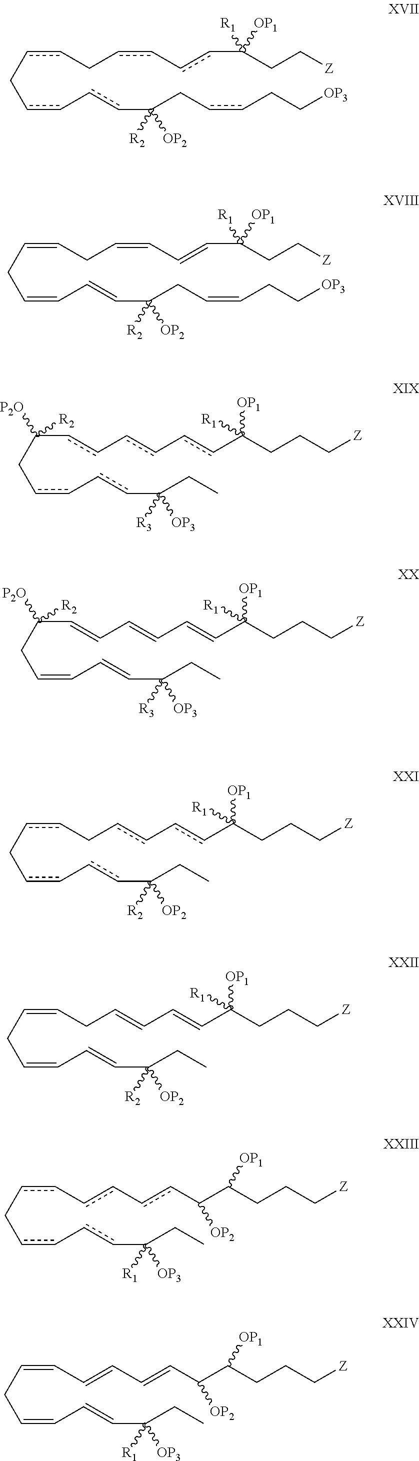

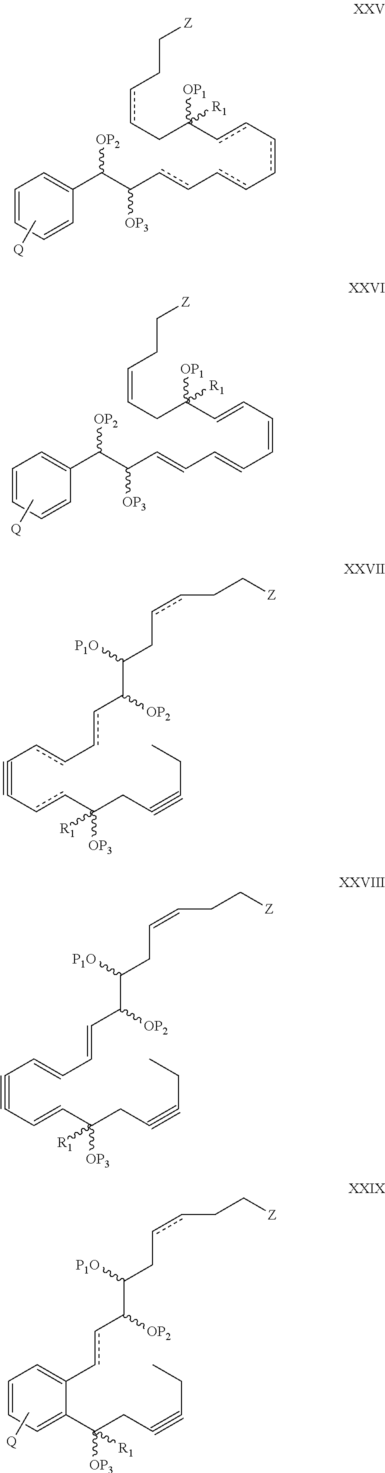

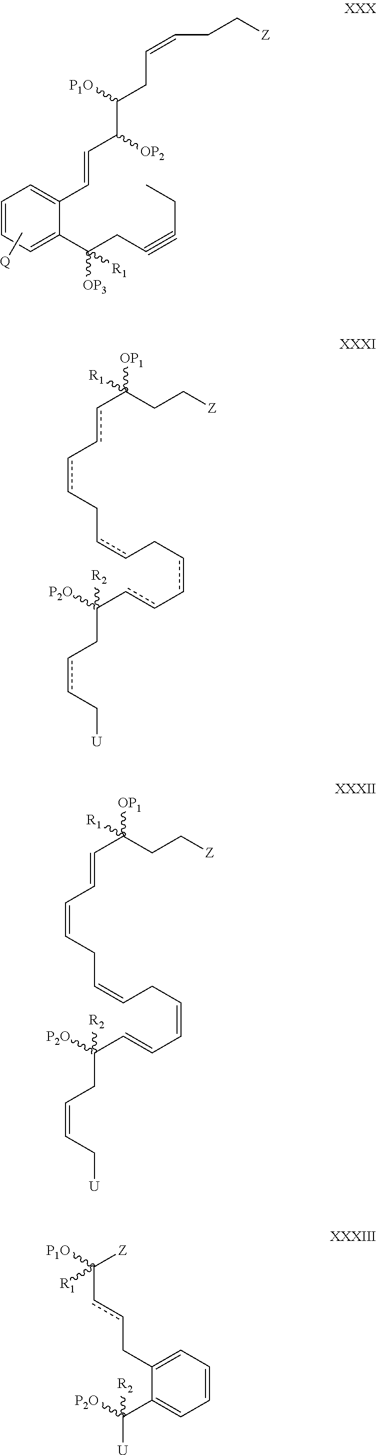

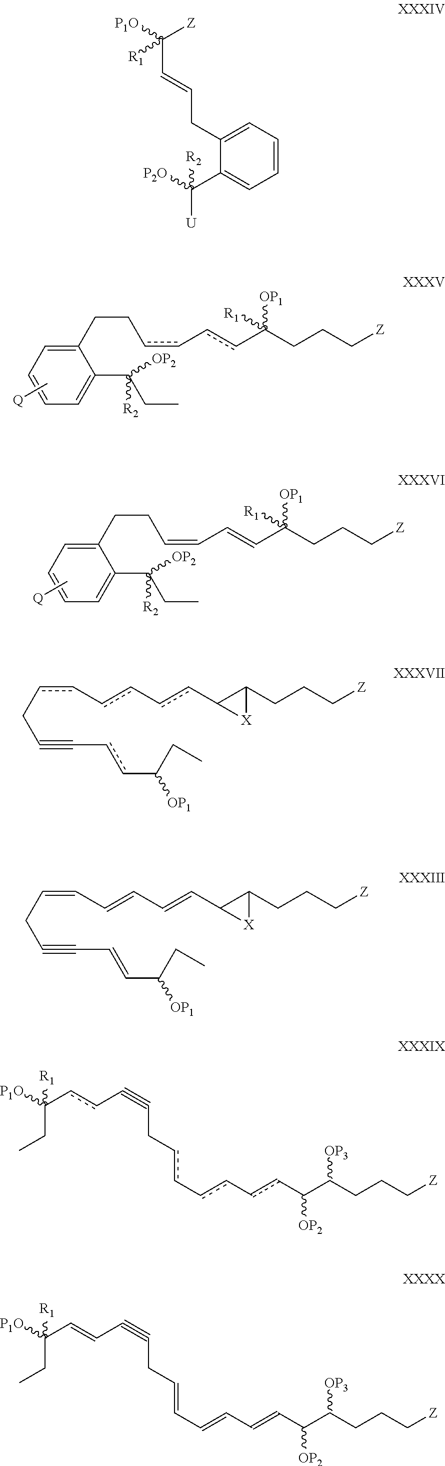

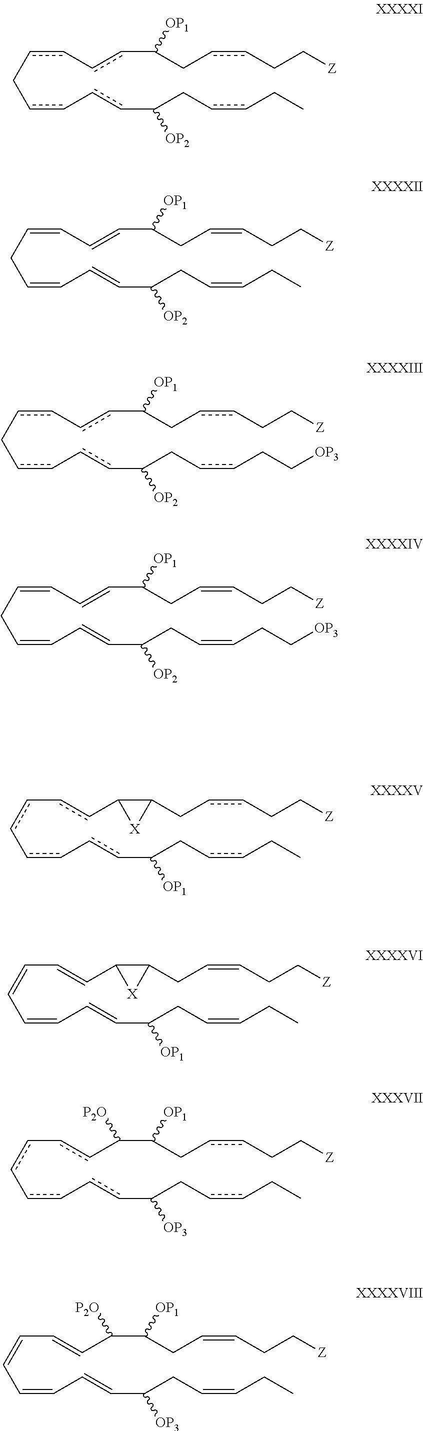

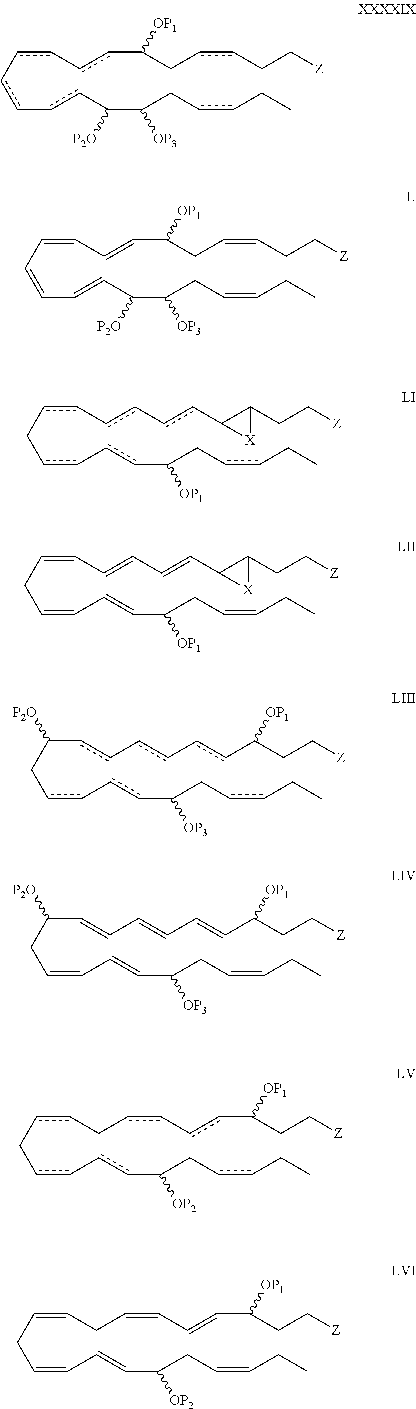

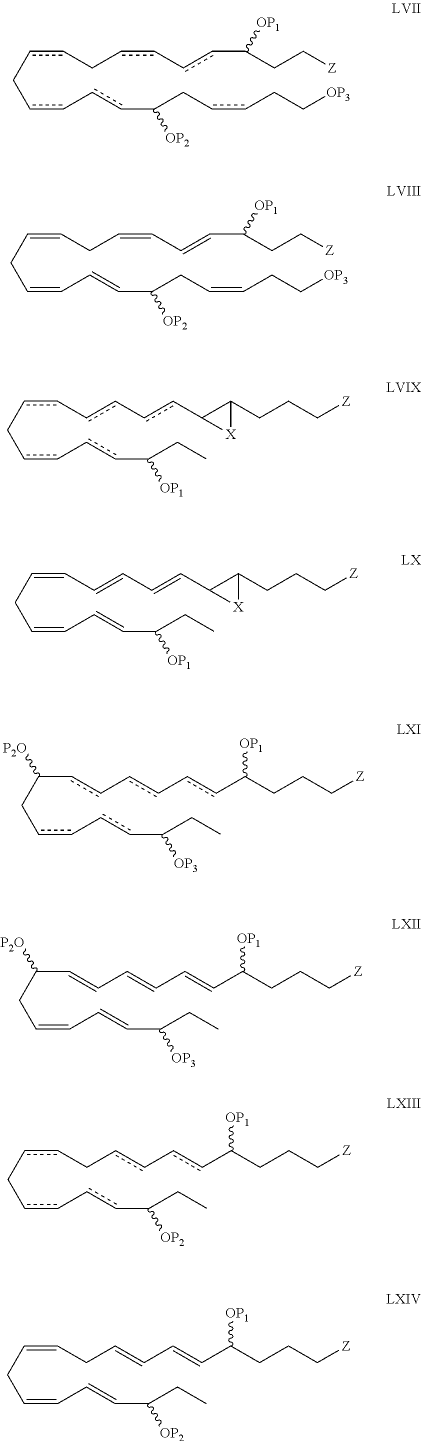

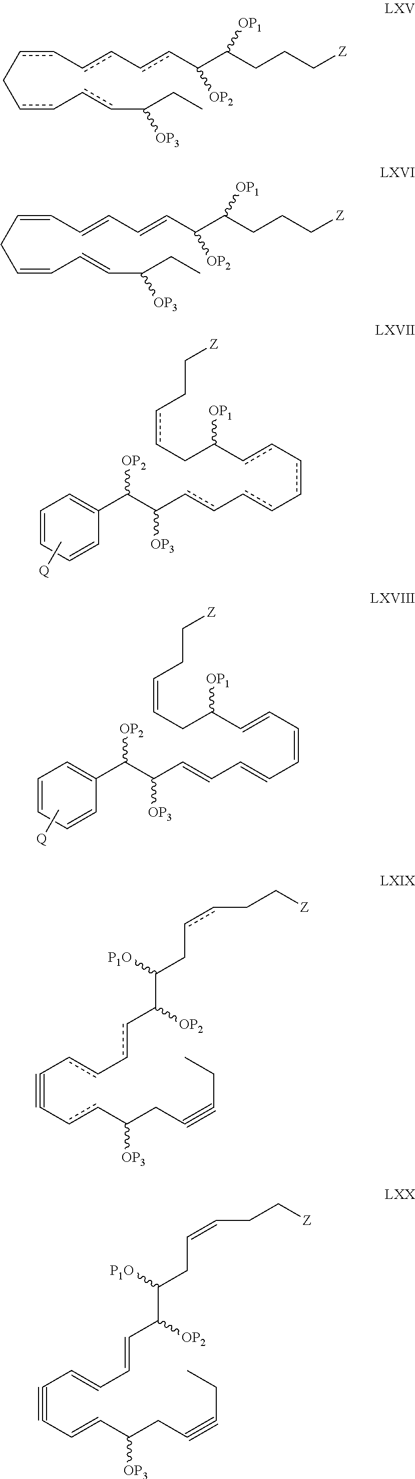

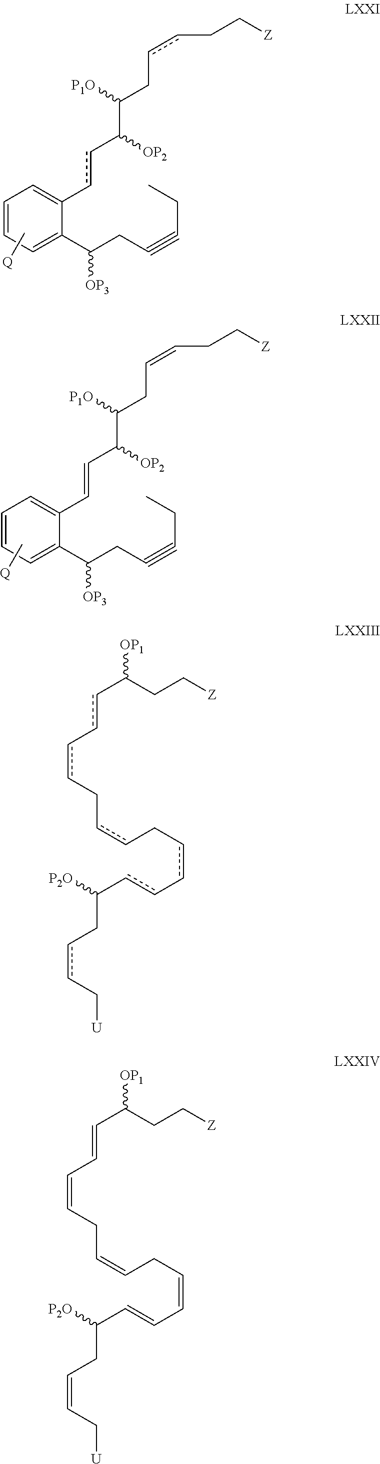

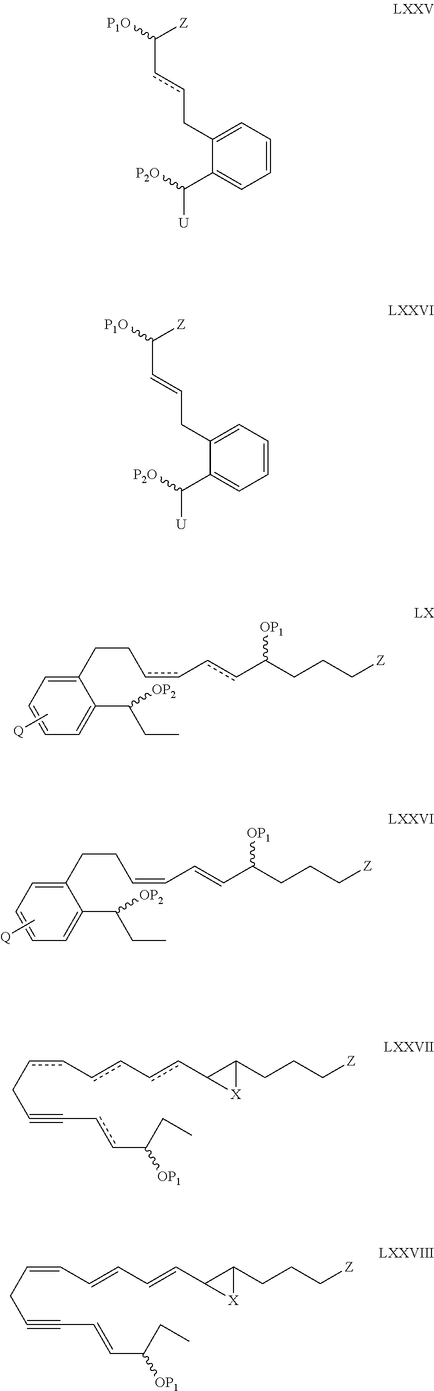

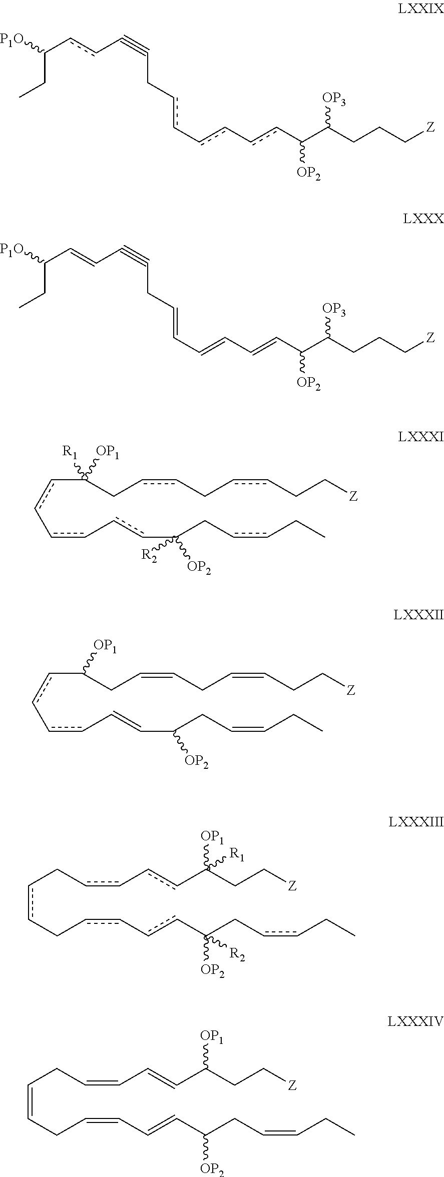

wherein a bond depicted as [symbol] represents either a cis or trans double bond;

wherein P₁, P₂ and P₃, if present, each individually are protecting groups, hydrogen atoms or combinations thereof;

wherein R₁, R₂ and R₃, if present, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof;

wherein Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;

each R^{a}, if present, is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b}, if present, is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}(NR^{a})]ₙNR^{c}R^{c};

each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;

each n, independently, if present, is an integer from 0 to 3;

each R^{d}, independently, if present, is a protecting group or R^{a};

in particular, Z is a carboxylic acid, ester, amide, thiocarbamate, carbamate, thioester, thiocarboxamide or a nitrile;

wherein X, if present, is a substituted or unsubstituted methylene, an oxygen atom, a substituted or unsubstituted nitrogen atom, or a sulfur atom;

wherein Q, if present, represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;

wherein U, if present, is a branched or unbranched, substituted or unsubstituted alkyl, alkenyl, alkynyl, cycloalkyl, aryl, alkoxy, aryloxy, alkylcarbonyl, aryl carbonyl, alkoxycarbonyl, aryloxycarbonyl, alkoxycarbonyloxy, and aryloxycarbonyloxy group;

and pharmaceutically acceptable salts thereof.

In certain embodiments, Z is a pharmaceutically acceptable salt of a carboxylic acid, and in particular is an ammonium salt or forms a prodrug.

In certain embodiments, P1, P2, and P3, if present, each individually are hydrogen atoms and Z is a carboxylic acid or ester. In other embodiments, X is an oxygen atom, one or more P's are hydrogen atoms, and Z is a carboxylic acid or ester. In still other embodiments, Q is one or more halogen atoms, one or more P's are hydrogen atoms, and Z is a carboxylic acid or ester.

In certain embodiments, R₁, R₂ and R₃, if present, are each individually lower alkyl groups, such as methyl, ethyl, and propyl and can be halogenated, such as trifluoromethyl. In one aspect, at least one of R₁, R₂ and R₃, if present, is not a hydrogen atom. Generally, Z is a carboxylic acid and one or more P's are hydrogen atoms.

In certain embodiments, when OP₃ is disposed terminally within the resolvin analog, the protecting group can be removed to afford a hydroxyl. Alternatively, in certain embodiments, the designation of OP₃ serves to denote that the terminal carbon is substituted with one or more halogens, i.e., the terminal C-18, C-20, or C-22 carbon, is a trifluoromethyl group, or arylated with an aryl group that can be substituted or unsubstituted as described herein. Such manipulation at the terminal carbon serves to protect the resolvin analog from omega P₄₅₀ metabolism that can lead to biochemical inactivation.

In certain embodiments, P₁, P₂, and P₃, if present, each individually are hydrogen atoms and Z is a carboxylic ester. In other embodiments, P₁, P₂, and P₃, if present, each individually are hydrogen atoms and Z is not carboxylic acid.

In one aspect, the compounds described herein are isolated and/or purified, in particular, compounds in which P₁, P₂, and P₃, if present, each individually are hydrogen atoms and Z is a carboxylic acid, are isolated and or purified.

In one aspect, the resolvins described herein that contain epoxide, cyclopropane, azine, or thioazine rings within the structure also serve as enzyme inhibitors that increase endogenous resolvin levels in vivo and block “pro” inflammatory substances, their formation and action in vivo, such as leukotrienes and/or LTB₄.

Another embodiment of the present invention is directed to pharmaceutical compositions of the novel compounds described throughout the specification useful to treat the conditions described herein.

The present invention also provides methods to treat the disease states and conditions described herein.

The present invention also provides packaged pharmaceuticals that contain the novel di- and tri-hydroxy EPA and DHA derivatives described throughout the specification for use in treatment with the disease states and conditions described herein.

It should be understood that throughout the specification, all compounds, including intermediates, can be isolated and purified by methods known in the art, such as distillation, chromatography, crystallization, filtration, HPLC, etc. The purity of the compound can be from about 80% to about 100%, in particular from about 85% to about 99.9%, more particularly from about 90% to about 99.5% and even more particularly from about 95% to about 99.9%.

“Alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. In preferred embodiments, the alkyl groups are C1-C6) alkyl.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl(isopropyl), cyclopropan-1-yl, etc.; butanyls such as butan-1-yl, butan-2-yl(sec-butyl), 2-methyl-propan-1-yl(isobutyl), 2-methyl-propan-2-yl(t-butyl), cyclobutan-1-yl, etc.; and the like. In preferred embodiments, the alkanyl groups are (C1-C6) alkanyl.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In preferred embodiments, the alkenyl group is (C2-C6) alkenyl.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In preferred embodiments, the alkynyl group is (C2-C6) alkynyl.

“Alkyldiyl” by itself or as part of another substituent refers to a saturated or unsaturated, branched, straight-chain or cyclic divalent hydrocarbon group having the stated number of carbon atoms (i.e., C1-C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group is C1-C6) alkyldiyl. Also preferred are saturated acyclic alkanyldiyl groups in which the radical centers are at the terminal carbons, e.g., methandiyl(methano); ethan-1,2-diyl(ethano); propan-1,3-diyl(propano); butan-1,4-diyl(butano); and the like (also referred to as alkylenos, defined infra).

“Alkyleno” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is C1-C6) or C1-C3) alkyleno. Also preferred are straight-chain saturated alkano groups, e.g., methano, ethano, propano, butano, and the like.

“Heteroalkyl,” Heteroalkanyl,” Heteroalkenyl,” Heteroalkynyl,” Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6) alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Acyclic Heteroatomic Bridge” refers to a divalent bridge in which the backbone atoms are exclusively heteroatoms and/or heteroatomic groups. Typical acyclic heteroatomic bridges include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6) alkyl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C5-C15) aryl, with (C5-C10) being even more preferred. Particularly preferred aryls are cyclopentadienyl, phenyl and naphthyl.

“Arylaryl” by itself or as part of another substituent refers to a monovalent hydrocarbon group derived by the removal of one hydrogen atom from a single carbon atom of a ring system in which two or more identical or non-identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl-naphthyl, binaphthyl, biphenyl-naphthyl, and the like. Where the number of carbon atoms in an arylaryl group are specified, the numbers refer to the carbon atoms comprising each parent aromatic ring. For example, (C5-C15) arylaryl is an arylaryl group in which each aromatic ring comprises from 5 to 15 carbons, e.g., biphenyl, triphenyl, binaphthyl, phenylnaphthyl, etc. Preferably, each parent aromatic ring system of an arylaryl group is independently a (C5-C15) aromatic, more preferably a (C5-C10) aromatic. Also preferred are arylaryl groups in which all of the parent aromatic ring systems are identical, e.g., biphenyl, triphenyl, binaphthyl, trinaphthyl, etc.

“Biaryl” by itself or as part of another substituent refers to an arylaryl group having two identical parent aromatic systems joined directly together by a single bond. Typical biaryl groups include, but are not limited to, biphenyl, binaphthyl, bianthracyl, and the like. Preferably, the aromatic ring systems are (C5-C15) aromatic rings, more preferably (C5-C10) aromatic rings. A particularly preferred biaryl group is biphenyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In preferred embodiments, the arylalkyl group is (C6-C21) arylalkyl, e.g., the alkanyl, alkenyl or alkynyl moiety of the arylalkyl group is (C1-C6) and the aryl moiety is (C5-C15). In particularly preferred embodiments the arylalkyl group is (C6-C13), e.g., the alkanyl, alkenyl or alkynyl moiety of the arylalkyl group is (C1-C3) and the aryl moiety is (C5-C10).

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), S(O)₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5-14 membered heteroaryl, with 5-10 membered heteroaryl being particularly preferred.

“Heteroaryl-Heteroaryl” by itself or as part of another substituent refers to a monovalent heteroaromatic group derived by the removal of one hydrogen atom from a single atom of a ring system in which two or more identical or non-identical parent heteroaromatic ring systems are joined directly together by a single bond, where the number of such direct ring junctions is one less than the number of parent heteroaromatic ring systems involved. Typical heteroaryl-heteroaryl groups include, but are not limited to, bipyridyl, tripyridyl, pyridylpurinyl, bipurinyl, etc. Where the number of atoms are specified, the numbers refer to the number of atoms comprising each parent heteroaromatic ring systems. For example, 5-15 membered heteroaryl-heteroaryl is a heteroaryl-heteroaryl group in which each parent heteroaromatic ring system comprises from 5 to 15 atoms, e.g., bipyridyl, tripuridyl, etc. Preferably, each parent heteroaromatic ring system is independently a 5-15 membered heteroaromatic, more preferably a 5-10 membered heteroaromatic. Also preferred are heteroaryl-heteroaryl groups in which all of the parent heteroaromatic ring systems are identical.

“Biheteroaryl” by itself or as part of another substituent refers to a heteroaryl-heteroaryl group having two identical parent heteroaromatic ring systems joined directly together by a single bond. Typical biheteroaryl groups include, but are not limited to, bipyridyl, bipurinyl, biquinolinyl, and the like. Preferably, the heteroaromatic ring systems are 5-15 membered heteroaromatic rings, more preferably 5-10 membered heteroaromatic rings.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is C1-C6) alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is C1-C3) alkyl and the heteroaryl moiety is a 5-10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula —NHR″ and “dialkylamine” refers to a group of the formula —NR″R″, where each R″ is independently an alkyl. As another example, “haloalkoxy” or “haloalkyloxy” refers to a group of the formula —OR′″, where R′″ is a haloalkyl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers.

Throughout the following descriptions, it should be understood that where particular double bonding is depicted, it is intended to include both cis and trans configurations. Exemplary formulae are provided with specific configurations, but for completeness, the double bonds can be varied. Not every structural isomer is shown in efforts to maintain brevity of the specification. However, this should not be considered limiting in nature. Additionally, where synthetic schemes are provided, it should be understood that all cis/trans configurational isomers are also contemplated and are within the scope and purview of the synthesis. Again, particular double bonding is depicted in exemplary manner.

In one embodiment, the analogs are designated as 10,17-diHDHAs. P₁ and P₂ are as defined above and can be the same or different. Z is as defined above and in particular can be a carboxylic acid, ester, amide, thiocarbamate, carbamate, thioester, thiocarboxamide or a nitrile. The broken double bond line, where noted, indicates that either the E or Z isomer is within the scope of the analog(s). In certain aspects, the chiral carbon atom at the 10 position (C-10) has an R configuration. In another aspect, the C-10 carbon atom has an S configuration. In still another aspect, the C-10 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate. In one example, the present invention includes 10,17S-docosatriene, 10,17S-dihydroxy-docosa-4Z,7Z,11E,13,15E,19Z-hexaenoic acid analogs such as 10R/S—OCH₃,17S-HDHA, 10R/S, methoxy-17S hydroxy-docosa-4Z,7Z,11E,13,15E,19Z-hexaenoic acid derivatives.

In certain embodiments, when P₁ and P₂ are hydrogen atoms and Z is a carboxylic acid, the compound is either isolated and/or purified.

In still yet another embodiment, the present invention pertains to diHDHA analogs that are designated as 4,17-diHDHAs. P₁, P₂ and Z are as defined above. P₁ and P₂ can be the same or different. In particular, Z can be a carboxylic acid, ester, amide, thiocarbamate, carbamate, thioester, thiocarboxamide or a nitrile. In certain aspects, the chiral carbon atom at the 4 position (C-4) has an R configuration. In another aspect, the C-4 carbon atom preferably has an S configuration. In still another aspect, the C-4 carbon atom is as an R/S racemate. Additionally, the chiral carbon atom at the 17 position (C-17) can have an R configuration. Alternatively, the C-17 carbon can have an S configuration. In still yet another aspect, the C-17 carbon can preferably exist as an R/S racemate.

In certain embodiments, when P ₁ and P₂ are hydrogen atoms and Z is a carboxylic acid, the compound is either isolated and/or purified.

For example, the present invention includes 4S,17R/S-diHDHA, 4S,17R/S-dihydroxy-docosa-5E,7Z,10Z,13Z,15E,19Z-hexaenoic acid analogs.

It should be understood that “Z” can be altered from one particular moiety to another by a skilled artisan. In order to accomplish this in some particular instances, one or more groups may require protection. This is also within the skill of an ordinary artisan. For example, a carboxylic ester (Z) can be converted to an amide by treatment with an amine. Such interconversion are known in the art.

In the EPA and DHA analogs, it should be understood that reference to “hydroxyl” stereochemistry is exemplary, and that the term is meant to include protected hydroxyl groups as well as the free hydroxyl group. In certain embodiments, the C-17 position has an R configuration. In other embodiment, the C-17 position has an S configuration. In other aspects, certain embodiments of the invention have an R configuration at the C-18 position.

In certain aspects of the present invention, ASA pathways generate R>S and therefore, 4S, 5R/S, 7S, 8R/S, 11R, 12R/S 16S, 17R. With respect to species generated from the 15-LO pathway the chirality of C-17 is S, C-16 R and C-10, preferably R.

The hydroxyl(s) in the EPA and DHA analogs can be protected by various protecting groups (P), such as those known in the art. An artisan skilled in the art can readily determine which protecting group(s) may be useful for the protection of the hydroxyl group(s). Standard methods are known in the art and are more fully described in literature. For example, suitable protecting groups can be selected by the skilled artisan and are described in Green and Wuts, “Protecting Groups in Organic Synthesis”, John Wiley and Sons, Chapters 5 and 7, 1991, the teachings of which are incorporated herein by reference. Preferred protecting groups include methyl and ethyl ethers, TMS or TIPPS groups, acetate (esters) or propionate groups and glycol ethers, such as ethylene glycol and propylene glycol derivatives.

For example, one or more hydroxyl groups can be treated with a mild base, such as triethylamine in the presence of an acid chloride or silyl chloride to facilitate a reaction between the hydroxyl ion and the halide. Alternatively, an alkyl halide can be reacted with the hydroxyl ion (generated by a base such as lithium diisopropyl amide) to facilitate ether formation.

The compounds can be prepared by methods provided in U.S. patent application Ser. No. 09/785,866, filed Feb. 16, 2001, entitled “Aspirin Triggered Lipid Mediators” by Charles N. Serhan and Clary B. Clish, Ser. No. 10/639,714, filed Aug. 12, 2003, entitled “Resolvins: Biotemplates for Novel Therapeutic Interventions” by Charles N. Serhan and PCT Applications WO 01/60778, filed Feb. 16, 2001, entitled “Aspirin Triggered Lipid mediators” by Charles N. Serhan and Clary B. Clish and WO 04/014835, filed Aug. 12, 2003, entitled “Resolvins: Biotemplates for Novel Therapeutic Interventions” by Charles N. Serhan, the contents of which are incorporated herein by reference in their entirety.

It should also be understood that for the EPA and DHA analogs, not all hydroxyl groups need be protected. One, two or all three hydroxyl groups can be protected. This can be accomplished by the stoichiometric choice of reagents used to protect the hydroxyl groups. Methods known in the art can be used to separate the di- or tri-protected hydroxy compounds, e.g., HPLC, LC, flash chromatography, gel permeation chromatography, crystallization, distillation, etc.

It should be understood that there are one or more chiral centers in each of the above-identified compounds. It should be understood that the present invention encompasses all stereochemical forms, e.g., enantiomers, diastereomers and racemates of each compound. Where asymmetric carbon atoms are present, more than one stereoisomer is possible, and all possible isomeric forms are intended to be included within the structural representations shown. Optically active (R) and (S) isomers may be resolved using conventional techniques known to the ordinarily skilled artisan. The present invention is intended to include the possible diastereiomers as well as the racemic and optically resolved isomers.

The resolvin analogs depicted throughout the specification contain acetylenic and/or ethylenically unsaturated sites. Where carbon carbon double bonds exist, the configurational chemistry can be either cis (Z) or trans (E) and the depictions throughout the specification are not meant to be limiting. The depictions are, in general, presented based upon the configurational chemistry of related DHA or EPA compounds, and although not to be limited by theory, are believed to possess similar configuration chemistry.

Throughout the specification carbon carbon bonds in particular have been “distorted” for ease to show how the bonds may ultimately be positioned relative one to another. For example, it should be understood that acetylenic portions of the resolvins actually do include a geometry of approximately 180 degrees, however, for aid in understanding of the synthesis and relationship between the final product(s) and starting materials, such angles have been obfuscated to aid in comprehension.

It should be understood that hydrogenation of acetylenic portions of the resolvin analog may result in one or more products.

It is intended that all possible products are included within this specification. For example, hydrogenation of a diacetylenic resolvin analog can produce up to 8 products (four diene products, i.e., cis, cis; cis, trans; trans, cis; trans, trans) if hydrogenation of both acetylenic portions is completed (this can be monitored by known methods) and four monoacetylene-monoethylene products (cis or trans “monoene”-acetylene; acetylene-cis or trans “monoene”. All products can be separated and identified by HPLC, GC, MS, NMR, IR.

Known techniques in the art can be used to convert the carboxylic acid/ester functionality of the resolvin analog into carboxamides, thioesters, nitrile, carbamates, thiocarbamates, etc. and are incorporated herein. The appropriate moieties, such as amides, can be further substituted as is known in the art.

In general, the resolvin analogs of the invention are bioactive as alcohols. Enzymatic action or reactive oxygen species attack at the site of inflammation or degradative metabolism. Such interactions with the hydroxyl(s) of the resolvin molecule can eventually reduce physiological activity as depicted below:

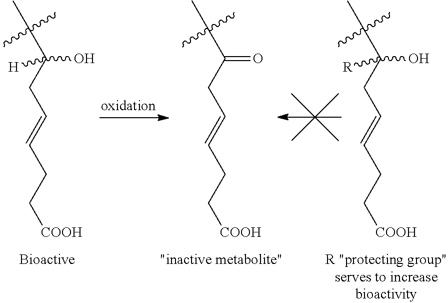

The use of “R” groups with secondary bioactive alcohols, in particular, serves to increase the bioavailability and bioactivity of the resolvin analog by inhibiting or diminishing the potential for oxidation of the alcohol to a ketone producing an inactive metabolite. The R “protecting groups” include, for example, linear and branched, substituted and unsubstituted alkyl groups, aryl groups, alkylaryl groups, phenoxy groups, and halogens.

Generally the use of “R protection chemistry” is not necessary with vicinal diols within the resolvin analog. Typically vicinal diols are not as easily oxidized and therefore, generally do not require such protection by substitution of the hydrogen atom adjacent to the oxygen atom of the hydroxyl group. Although it is generally considered that such protection is not necessary, it is possible to prepare such compounds where each of the vicinal diol hydroxyl groups, independently, could be “protected” by the substitution of the hydrogen atom adjacent to the oxygen atom of the hydroxyl group with an “R protecting group” as described above.

The term “tissue” is intended to include intact cells, blood, blood preparations such as plasma and serum, bones, joints, muscles, smooth muscles, and organs.

The term “subject” is intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient, i.e., at least one EPA or DHA analog, in combination with a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention. Such solutions are useful for the treatment of conjunctivitis.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Intravenous injection administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 0.1 to about 40 mg per kg per day. For example, between about 0.01 microgram and 20 micrograms, between about 20 micrograms and 100 micrograms and between about 10 micrograms and 200 micrograms of the compounds of the invention are administered per 20 grams of subject weight.

If desired, the effective daily dose of the active compound may be administered as two, three, four, five, six or more sub-doses administered separately at appropriate intervals throughout the day, optionally, in unit dosage forms.

The pharmaceutical compositions of the invention include a “therapeutically effective amount” or a “prophylactically effective amount” of one or more of the EPA or DHA analogs of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the EPA or DHA analog may vary according to factors such as the disease state, age, sex, and weight of the individual, and the ability of the therapeutic compound to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the EPA or DHA analog and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a EPA or DHA analog of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Delivery of the EPA or DHA analogs of the present invention to the lung by way of inhalation is an important method of treating a variety of respiratory conditions (airway inflammation) noted throughout the specification, including such common local conditions as bronchial asthma and chronic obstructive pulmonary disease. The EPA or DHA analogs can be administered to the lung in the form of an aerosol of particles of respirable size (less than about 10 μm in diameter). The aerosol formulation can be presented as a liquid or a dry powder. In order to assure proper particle size in a liquid aerosol, as a suspension, particles can be prepared in respirable size and then incorporated into the suspension formulation containing a propellant. Alternatively, formulations can be prepared in solution form in order to avoid the concern for proper particle size in the formulation. Solution formulations should be dispensed in a manner that produces particles or droplets of respirable size.

Once prepared an aerosol formulation is filled into an aerosol canister equipped with a metered dose valve. The formulation is dispensed via an actuator adapted to direct the dose from the valve to the subject.

Formulations of the invention can be prepared by combining (i) at least one EPA or DHA analog in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

Aerosol canisters equipped with conventional valves, preferably metered dose valves, can be used to deliver the formulations of the invention. Conventional neoprene and buna valve rubbers used in metered dose valves for delivering conventional CFC formulations can be used with formulations containing HFC-134a or HFC-227. Other suitable materials include nitrile rubber such as DB-218 (American Gasket and Rubber, Schiller Park, Ill.) or an EPDM rubber such as Vistalon™ (Exxon), Royalene™ (UniRoyal), bunaEP (Bayer). Also suitable are diaphragms fashioned by extrusion, injection molding or compression molding from a thermoplastic elastomeric material such as FLEXOMER™ GERS 1085 NT polyolefin (Union Carbide).

Formulations of the invention can be contained in conventional aerosol canisters, coated or uncoated, anodized or unanodized, e.g., those of aluminum, glass, stainless steel, polyethylene terephthalate.

The formulation(s) of the invention can be delivered to the respiratory tract and/or lung by oral inhalation in order to effect bronchodilation or in order to treat a condition susceptible of treatment by inhalation, e.g., asthma, chronic obstructive pulmonary disease, etc. as described throughout the specification.

The formulations of the invention can also be delivered by nasal inhalation as known in the art in order to treat or prevent the respiratory conditions mentioned throughout the specification.

While it is possible for a compound of the present invention to be administered alone, it is preferable to administer the compound as a pharmaceutical composition.

The invention features an article of manufacture that contains packaging material and a EPA or DHA analog formulation contained within the packaging material. This formulation contains an at least one EPA or DHA analog and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference. Suitable EPA analogs and DHA analogs are described herein.

More specifically, the invention features an article of manufacture that contains packaging material and at least one EPA or DHA analog contained within the packaging material. The packaging material contains a label or package insert indicating that the formulation can be administered to the subject to asthma in an amount, at a frequency, and for a duration effective treat or prevent symptoms associated with such disease states or conditions discussed throughout this specification.

### EXAMPLES

Various exemplary embodiments of the devices, compounds and methods as generally described above according to this invention, will be understood more readily by reference to the following examples, which are provided by way of illustration and are not intended to be limiting of the invention in any fashion.

### Example 1

### Animals

All animals used in the present study were male FVB mice (Charles River Laboratories, Wilmington, Mass.) that were 6- to 8-weeks-old (weighing 20-25 g). They were maintained in a temperature and light-controlled environment, and had unlimited access to food (Laboratory standard rodent diet 5001 (Lab Diet, St. Louis, Mo.), containing EPA 1.5%, DHA 1.9% of total fatty acids, and tap water. Experiments were performed in accordance with the Harvard Medical School Standing Committee on Animals guidelines for animal care (Protocol no. 02570).

### Example 2

### Chemicals

RvD1, 17-(R/S)-methyl RvD1, and RvE1 were each synthesized by total organic synthesis for these experiments from starting materials of known chirality in enantiomerically and geometrically pure form (Dr. Nicos Petasis, Organic Synthesis Core, NIH P50-DE-016191). The 19-p-fluorophenoxy-RvE1 methyl ester was prepared in a stereochemically pure form as in ref. 30. Physical and spectroscopic properties matching biogenic and synthetic RvD1 and RvE1 were as reported^{24, 31}. The integrity of the synthetic resolvins and their analogs was assured by monitoring the physical properties with LC-UV-tandem mass spectrometry just prior to evaluating their biological activities.

### Example 3

### Murine Peritonitis

Male FVB mice were anesthetized with isoflurane (Hospira Inc, Lake Forest, Ill.). Both 1 μg d₅-DHA (4Z,7Z,10Z,13Z,16Z,19Z-docosahexaenoic-21,21,22,22,22-d₅ acid: Cayman Chemical, Ann Arbor, Mich.) and 1 μg d₅-EPA (5Z,8Z,11Z,14Z,17Z-eicosapentaenoic-19,19′,20,20,20-d₅ acid: Cayman Chemical, Ann Arbor, Mich.) in 100 μl of 2% ethanol (v/v) in saline or vehicle alone were administered by bolus tail vein injection. Approximately five minutes later, peritonitis was initiated with intraperitoneal administration of 1 mg of zymosan A (Sigma Aldrich, St. Louis, Mo.). At indicated time intervals, mice were euthanized with an overdose of isoflurane, and peritoneal exudates were collected by lavage with 5 ml of DPBS without either Ca²⁺ or Mg²⁺. Exudate cells and supernatants were separated by centrifugation (10 min, 1000 rpm, 4° C.), aliquots of supernatants were collected, and 4 volumes of cold acetone were added to precipitate proteins. Serum albumin and total protein levels were determined³².

Briefly, FVB mice (6-8 wk) received d₅-EPA and d₅-DHA i.v. (1 μg/mouse of each) (FIG. 4D, top panel) just before i.p. challenge with zymosan A (1 mg/ml/mouse) (FIG. 4D, middle panel). Peritoneal exudates were collected at 0, 2, 4, 12, 24 and 48 h and taken to solid phase extraction. Eluate fractions were treated with diazomethane and taken to GC-MS. Results represent the mean±SEM from n=3, separate mice. Mass spectra of d₅-EPA methyl ester and d₅-DHA methyl ester. d₅-EPA and d₅-DHA were derivatized into methyl esters and submitted to GC-MS for analysis shown in FIGS. 4A-C.

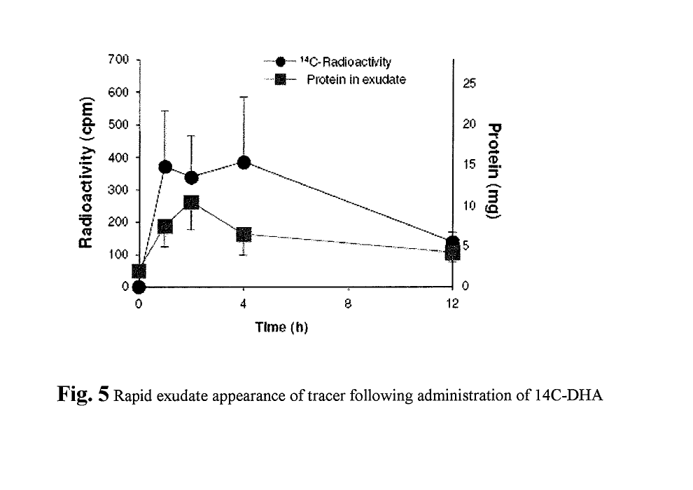

For tracer experiments, mice were fasted overnight (˜18 h) before 500 nCi of [1-¹⁴C]-DHA (docosahexaenoic acid 4,7,10,13,16,19-[1-¹⁴C], American Radiolabeled Chemicals Inc, St. Louis, Mo.) was injected via the tail vein. At indicated time intervals, exudates were collected (vide supra) and the radioactivity was determined using a scintillation counter (Multi-purpose scintillation counter LS6500, Beckman Coulter, Fullerton, Calif.). This data is illustrated in FIG. 5.

### Example 4

### Mass Spectrometric Analysis of Deuterium Labeled EPA and DHA

Upon collection of peritoneal exudates, 2 volumes of cold methanol and internal standard (500 ng/sample, trans-parinaric acid, Molecular Probe, Eugene, Oreg.) were added to exudates, which were stored at −20° C. for 1 hr. After centrifugation, samples were diluted and applied to C18 cartridge columns (Extract-Clean EV SPE, Alltech, Deerfield, Ill.). Lipids were extracted using hexane and methyl formate and each fraction was collected and its unesterified fatty acids converted to corresponding methyl ester using diazomethane treatment³⁴. The esters were suspended in hexane, and then injected in GC-MS. Electron-impact GC-MS was carried out using an HP 6890 GC system with HP5973 Mass Selective Detector (Hewlett-Packard) equipped with an HP-5MS capillary column (0.25 mm ID×30 m, 0.25 μm, Agilent Technologies Inc., Wilmington, Del.) operating at a mass range from m/z 70 to 800. The ionization voltage was 70 eV and the ion source temperature was 230° C. Chromatography was carried out using a column temperature maintained at 150° C. for 2 min and then programmed to increase 10° C./min up to 230° C., 5° C./min up to 280° C., and then maintained at 280° C. for 10 min. EPA and DHA were separated and the area beneath each chromatographic peak of their deuterium labels was obtained by integration using a Chemstation integrator Version D.02.00.275 (Agilent Technologies Inc). For an internal standard, parinaric acid was selected since it exists in plants and its spectrum does not overlap with those of endogenous fatty acids for diagnostic ions. The inventors selected the ω-3 ion ((CH)₃CH₂(CH)₂CD₂CD₃=113) for both d₅-EPA (FIG. 4A) and d₅-DHA (FIG. 4B) methyl ester and M⁺(290) for methyl parinate (FIG. 4C). Identification was conducted by examining and retention times as in FIGS. 4A-C (d₅-EPA-methyl ester: 11.7 min FIG. 4A, inset, methyl-parinarate; 13.1 min (FIG. 4C, and d₅-DHA; 13.9 min, FIG. 4B, inset). Calibration curves were obtained for each: d₅-EPA; y=0.0016x+0.0313 (r²=0.9954, 50-500 ng/ml), d₅-DHA; y=0.0015x-0.0524 (r²=0.9809, 50-500 ng/ml).

### Example 5

### Differential Counts and FACS Analysis

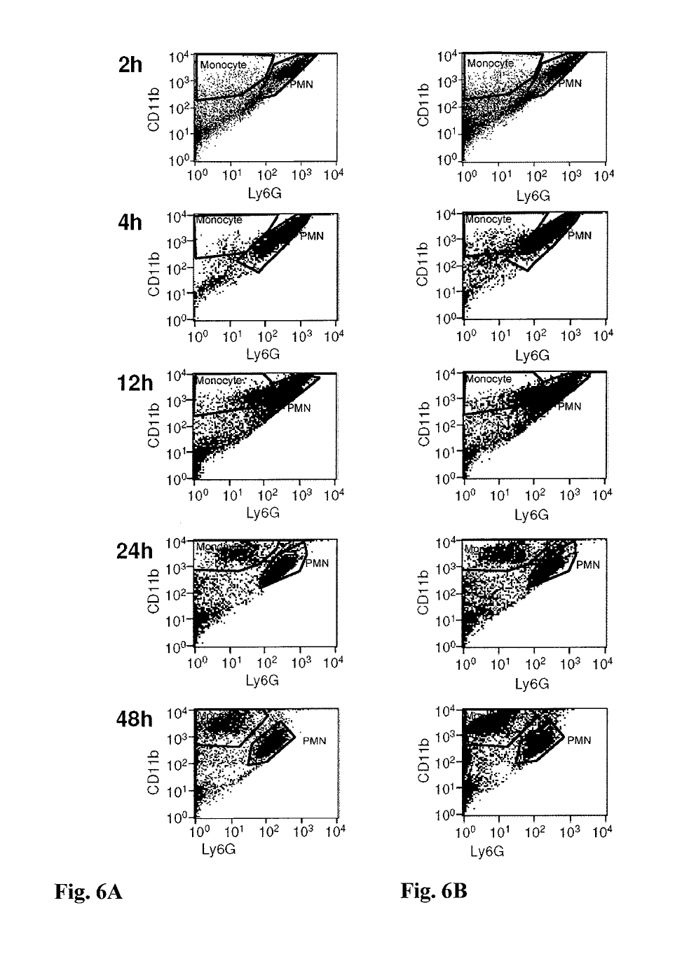

Aliquots of lavage cells were assessed for total and differential leukocyte counts via light microscopy to identify individual cell types (i.e., neutrophil, monocyte, etc.). For flow cytometry analysis, aliquots of 0.5×10⁶ cells were stained with 0.25 μg FITC-conjugated anti-mouse F4/80, 0.1 μg PE-conjugated anti-mouse Ly-6G, and 0.1 μg PerCP-Cy5.5-conjugated anti-mouse CD11b, or 0.1 μg PE-conjugated Rat IgG2c,κ isotype control as a background stain. Cells were washed and analyzed using FACSort (BD Biosciences, San Jose, Calif.)³³. Data are shown in FIGS. 6A and B. These results indicate that a large portion of the lavage cells resulting from the zymosan induced inflammation are PMN and monocytes.

### Example 6

### Ischemia-Reperfusion-Induced Second-Organ Injury

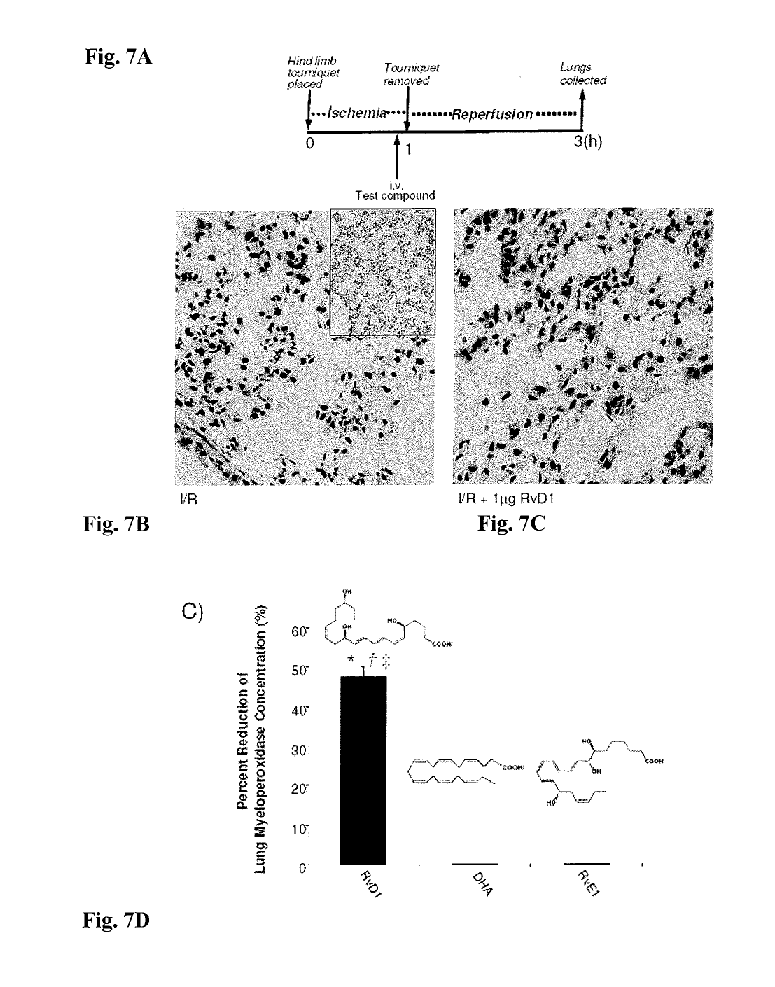

To identify the effects of the resolvins and protectins on inflammation resolution, ischemia-reperfusion induced second-organ injury experiments were designed and are illustrated in FIGS. 7A-D. Mice were anesthetized by intraperitoneal injection of pentobarbital (80 mg kg⁻¹, Nembutal sodium solution NDC 0074-3778-04). Hind-limb ischemia was initiated using tourniquets consisting of a rubber band placed on each hind limb as in ref.³⁵. Mice were subjected to hind limb ischemia for 1 h, after which the tourniquets were removed to initiate reflow-reperfusion. Resolvins, related compounds and analogs were each administered at 1 μg/mouse (i.e., DHA, RvD1, RvD1 methyl ester, 17-(R/S)-methyl-RvD1 methyl ester, RvE1, or 19-p-fluorophenoxy-RvE1 methyl ester) in vehicle (5 μl ethanol in 120 μl sterile saline) or vehicle alone. They were administered intravenously to the tail vein ˜5 min before the start of the reperfusion period. At the end of this reperfusion period (2 h), the mice were euthanized with an overdose of anesthetic and the lungs were quickly harvested, frozen in liquid nitrogen, and stored at −80° C. The right lungs were homogenized from individual mice and centrifuged, and the tissue levels of myeloperoxidase (MPO) (a leukocyte marker enzyme) in the resulting supernatants, for each experimental protocol, were determined using a mouse MPO enzyme-linked immunosorbent assay (Hycult biotechnology, Cell Sciences, Uden, The Netherlands).

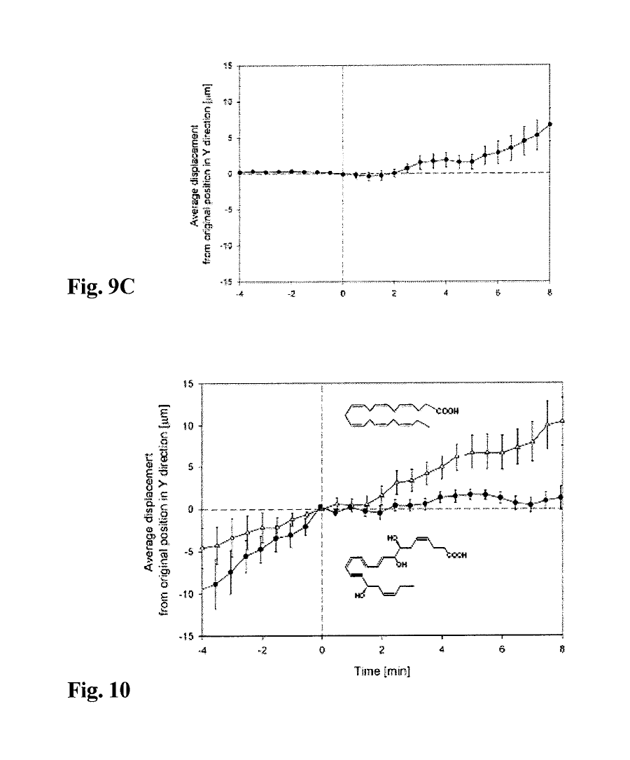

Briefly, as illustrated in the time-course shown in FIG. 7A, hind-limb ischemia was induced in mice by creating tourniquets using a rubber band on each hind limb. After 1 h, the tourniquets were removed and reperfusion ensued. Test compounds (1 μg of DHA, RvD1, RvE1) in vehicle were administered intravenously 5 min before the start of the reperfusion period. At the end of the reperfusion period (2 h), lungs were collected and MPO concentrations were determined by ELISA. FIGS. 7B and C are representative images from histological analysis. FIG. 9B is a photomicrograph showing a hematoxylin/eosin-stained lung from ischemia/reperfusion second organ injury (control). FIG. C shows hematoxylin/eosin-stained lung from 1 μg RvD1 i.v. injection to ischemia/reperfusion second organ injury followed by i.v. administration of RvD1. The results are quantified in FIG. 7D and indicate RvD1 significantly reduced the MPO concentration. Mean±SEM (n=3-5, control; n=12). RvD1, 47.5±2.4, n=5; DHA, 0±7.7, n=4; RvE1, 0±9.3, n=6. *, significantly different from values obtained with vehicle, p<0.02. †, significantly different from values obtained with DHA, p<0.01. ‡, significantly different from values obtained with RvE1, p<0.002.

### Example 7

### From Circulation to Inflammatory Exudates

To determine whether circulating unesterifed ω-3 fatty acids are available to evolving inflammatory exudates, the inventors administered intravenous deuterium labeled ω-3 fatty acids, e.g., d₅-EPA and d₅-DHA (FIG. 4A-B), to mice with a localized inflammation, namely peritonitis. The magnitude of the inflammatory insult was selected to be a self-limited spontaneous resolving peritonitis³⁷ to monitor the potential presence of deuterium labeled fatty acids in the inflammatory exudates. Both deuterium labeled EPA and DHA were identified within the local inflammatory exudates. As shown in FIG. 4C (upper panel), both d₅-EPA and d₅-DHA rapidly appeared in exudates during the time course of initiation of inflammation. For example, d₅-EPA reached maximum levels at 4 h and d₅-DHA was maximum at 2 h. The levels of both unesterified d₅-EPA and d₅-DHA gradually declined at 24 h. In this spontaneous resolution system, maximum inflammation as defined by maximum PMN infiltrates within the exudates was at ˜4 h and the resolution phase ranged between 12-24 h.

As discussed, PMN infiltration into the exudates was monitored. By definition, these exudates contain serum proteins³⁸ that were determined throughout the time course within the peritoneal exudates (FIG. 4C lower panel, cf. ref.³⁹). Of interest, the time course of both d₅-EPA and d₅-DHA paralleled the appearance and increase in protein in these inflammatory exudates. Their presence also coincided with PMN infiltration (FIG. 4C). At 48 h, both d₅-EPA and d₅-DHA levels were significantly greater than at 24 h. Thus, EPA and DHA, the precursors for resolvins and protectins, rapidly appeared within these developing inflammatory exudates from peripheral circulation.

The inventors used a second approach to verify that DHA, i.e., a representative ω-3 fatty acid, indeed appears in exudates from peripheral circulation (FIG. 5). To this end, radiolabeled ω-3 fatty acid tracer of DHA was administered i.v., and DHA and its products in exudates was monitored. To address this, the recovery of ¹⁴C-DHA and the total protein amount in murine exudates was determined. In order to reduce potential and immediate influences of diet, these experiments were performed with mice that were fasted overnight before receiving intravenous ¹⁴C-DHA. Exudates were collected at 1, 2, 4 and 12 h. At 1 h, ¹⁴C label had already reached maximal levels and protein levels appeared to parallel the ¹⁴C radioactivity profile. These results suggest that ¹⁴C-DHA and its products rapidly appear coincident with protein and increases in PMN infiltration within the exudate site of inflammation. Thus, they confirm that tracer levels of ¹⁴C-DHA and its products rapidly appeared within the exudates during early initiation of the inflammatory response in vivo (FIG. 5). Of note, these tracer levels of DHA did not reduce leukocyte trafficking as monitored by FACS analysis of the exudates (see FIGS. 6A and B).

### Example 8

### Resolvins are Organ Protective In Vivo

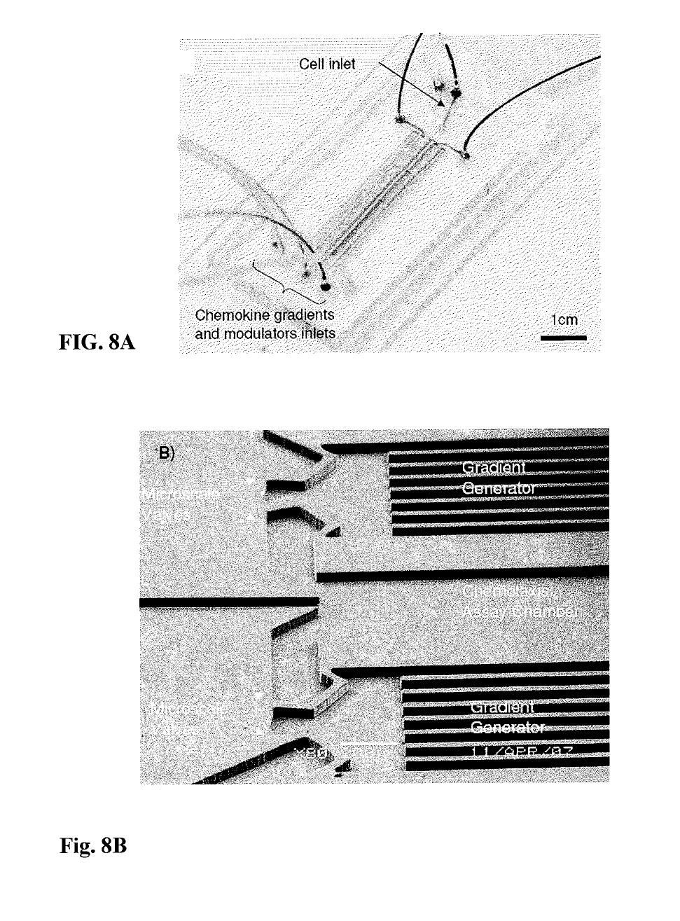

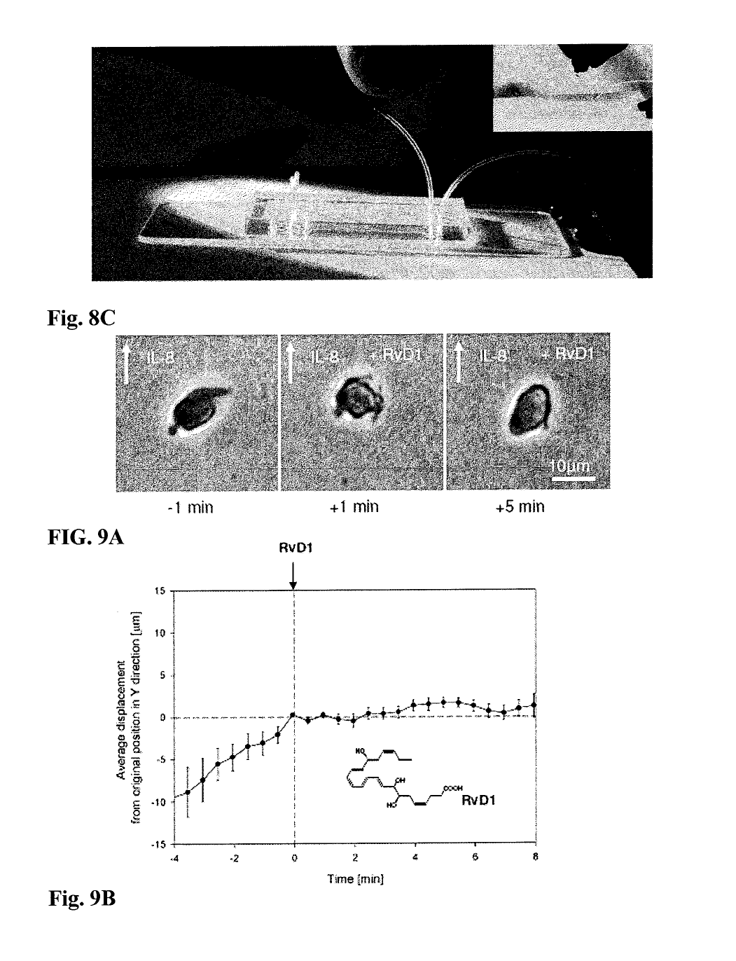

Ischemia/reperfusion is a well-appreciated pathophysiologic mechanism of organ injury and local tissue damage that can be initiated by excessively activated PMN. This system in the mouse, models the second organ injury observed in humans following tourniquet release of vessels in surgery involving, for example, extremities^{41, 42}. Remote or second organ injury can also occur when blood vessels are occluded, or during certain surgical procedures that create local ischemia and remote organ injury that has many features of rapid, acute inflammation and tissue damage³⁵. On release of the tourniquet occlusion, reflow is initiated and activated PMN rapidly infiltrate secondary organs, causing damage³⁵. Here, the inventors evaluated RvD1 in a remote organ injury system, i.e., hind-limb ischemia/reperfusion, to assess whether RvD1's ability to inhibit PMN migration, in vitro, in microchambers (FIGS. 8A-C, 9-11) can be predictive of their effect to reduce PMN mediated tissue and organ damage in vivo. As demonstrated by the experiments described in EXAMPLE 6 and represented by FIGS. 7A-D, PMN accumulation in the lung was assessed by monitoring increases in both tissue histology and tissue MPO, a leukocyte marker enzyme. The extent of lung tissue injury is associated with PMN activation and organ infiltration³⁵.

### Example 9

### Fabrication of Microfluidic Chemotaxis Device

A new microvolume chemotaxis device was designed using polyethylene glycol (PEG) that allows testing the chemotactic function of neutrophils and their responses to these novel lipid mediators immediately after being separated from whole blood within the device. One exemplary embodiment of the device is illustrated in FIGS. 8A-C. In this embodiment, the microvolume chemotaxis device with microstructured valves was fabricated using “lab-on-chip” microfabrication technologies³⁶. Briefly, two silicon wafers were coated with a 50-μm-thick layer of SU8 photoresist (MicroChem, Newton, Mass.). After exposure to UV light through a mylar mask in a mask, aligner and development following manufacturer recommendations, 50 μm-tall features were produced on top of the silicon wafers. Subsequently, poly (dimethylsiloxane) (PDMS, Dow Corning, Midland, Mich.) was prepared by mixing two components in 10:1 ratio, as recommended by the manufacturer. The wafer with network structures was coated with a thin film of PDMS by spinning in a spinner at 1000 rpm for 30 seconds. The wafer with the control structures was placed in a larger Petri dish and covered with a 4-mm-thick layer of PDMS. The PDMS was cured by placing the two wafers overnight in a 65° C. oven. Thicker sections of PDMS were removed from the wafer, cut to size, and holes punched using a sharpened needle (Small Parts, Miami Lakes, Fla.). The pieces and the wafer with the thin PDMS film were next treated with oxygen plasma (March, Concord, Calif.), aligned and bonded together on a 75° C. hot plate. After bonding, the PDMS was removed from the wafer, cut again and more holes punched through the two layers of PMDS. Finally, the two-layer PDMS constructs were then exposed to oxygen plasma and bonded on glass slides (Fisher Scientific, Pittsburgh, Pa.). In order to avoid the bonding of the valve membrane to the underlying glass, a vacuum was applied to the control channels. After the bonding of the main PDMS pieces, the lifted microstructured valves were moved up and down a few times. The successive contact and peeling of the PDMS on the glass rendered the microstructured membrane surface inactivated, preventing further bonding and assuring the correct operation ability of the microscale valve structure, shown in FIGS. 8A-C.

FIGS. 8A-8C illustrate one exemplary embodiment of the microfluidic chemotaxis device according to the invention. As shown in FIG. 8B, the microfluidic chemotaxis device includes two gradient generators. In the embodiment of the invention illustrated in FIG. 8B, the gradient generators further include network channels. As illustrated, the gradient channels flank a single chemotaxis assay chamber. Each gradient generator has two-pair of microscale valves. one pair leading to the gradient generators and one pair leading to the chemotaxis assay chamber. As shown in FIG. 8A a pair of modulator inlets leads to the outside of each of the microvalves leading to the gradient generator and a pair of chemokine gradient inlets leads to the chemotaxis chamber side of the second-pair of microscale valves. A cell inlet is further provided at the opposite end of the microvolume chemotaxis device. The device is optimized for visualization of the effects of chemotaxis on the cells captured in the device.

In other exemplary embodiments, the microfluidic chemotaxis device can be constructed as described. The following using the following reagents were used: Acrylate solution: 3-(trimethoxysilyl)-propyl acrylate (92%, Aldrich, 475149), 50 mcL/ml acetone; PEG solution: Poly(ethylene glycol)diacrylate (Aldrich, 437441) with 1% photo initiator (2,2-dimethoxy-2-phenyl-acetophenone, Aldrich, 19611-8); Acetone anhydrous; ddH2O. Briefly, To the chamber of freshly O₂ plasma cleaned device is added 5% (v/v) acrylate in acetone using 1 ml syringe with modified 200 mcL pipette tip. Wait for 5 mins. if necessary, more acrylate solution was added to keep the chamber wet from time to time. The chamber was rinsed with acetone, and air dried. PEG was added slowly with 1% photo initiator to the chamber. Do not let the PEG over-fill the out-let (it will be enough as long as the chamber is filled.) Wait for 15 min. Then using a 1 ml syringe with 200 mcl modified pipette tip to remove the PEG from the outlet of the chamber with air (syringe piston starts from 0.7 ml slowly move to 0.5 ml and wait. Air into the chamber can be seen after 5 to 15 sec, which depends on how much PEG is in the outlet.). A 1 ml syringe was then used with a modified pipette tip slowly add 5% H₂O in acetone (syringe piton moves from 0.6 ml to 0.5 ml, and wait) to the chamber from the inlet of the chamber. The excess PEG was removed with water in acetone. The device was rinsed an excess of acetone (2 to 3 tips) and air dried.

After assembly, different sections of the device were chemically modified for specific functionalities. The surfaces of the network channels in the gradient generator section (FIG. 8B) were modified with PEG to prevent adsorption of the lipid mediators to the PDMS surfaces. The protocol involved treating the PDMS surface with 3-(trimethoxysilyl)-propyl acrylate (5% in acetone, Sigma-Aldrich, St. Louis, Mo.), followed by three washes with acetone, and followed by poly(ethylene glycol)methyl ether acrylate (10% in acetone, Sigma-Aldrich) with 1% photo initiator (2,2-dimethoxy-2-phenyl-acetophenone, Sigma-Aldrich). After exposure to 352 nm uv light (XX-15BLB, UVP, Upland, Calif.) for 5 seconds, the devices were washed three times with acetone, and dried with nitrogen. After this surface modification procedure, the devices were stored in the refrigerator for up to a week. The surface of the chemotaxis chamber, where neutrophils were captured and assessed for their chemotactic responses, were modified by physical adsorption of P-selectin (10 ng/mL for 30 minutes, R&D Systems, Minneapolis, Minn.) immediately before use. The separation between the different sections of the device during the surface modification was achieved by keeping the valves between the main channels and the gradient generators closed.

### Example 10

### Leukocyte Motility and Chemotaxis

For these experiments, the device chambers were purged with Hanks' balanced salt solution buffer (HBSS, Sigma-Aldrich) with 0.1% human serum albumin (HSA, Sigma-Aldrich), with care to remove all bubbles. Four syringes, two with buffer, one with IL-8 chemoattractant (R&D Systems) and one with the lipid mediator, i.e., RvD1 or other related compounds tested in HBSS were placed in syringes (1 mL) with a syringe pump set for 0.1 μL/min. The flow stabilized for 3 min, with the microscale valves between the chemotaxis chamber and gradient generators closed (see FIG. 8B). Approximately 10 μL of capillary blood was collected from healthy volunteers by finger-prick using a BD genie lancet (Becton, Dickinson and Company, Franklin Lakes, N.J.). The whole blood was then quickly mixed with heparin (10 μL) in a syringe tip (30G, Small Parts) and, after opening the cell inlet valve, slowly pushed through the tubing (Tygon, Small Parts) into the microvolume chemotaxis device (FIG. 8C). The blood stayed in the main channel for 3 minutes and then the valve opened for the chemokine gradient generator. The flow removed the majority of red blood cells and other cells were not weakly attached, thus allowing direct observation of the neutrophils captured on the surface of the chamber. After 10-15 minutes, the gradient was switched to the gradient containing the chemokine along or either DHA or RvD1. The migration of neutrophils in the chemokine gradient and their response to RvD1 or native DHA were recorded with a video and/or CCD camera and cell migration was analyzed using the cell tracking function in Metamorph (Molecular Devices, Sunnyvale, Calif., USA). At least a dozen individual cells per condition were tracked and analyzed for displacement in the direction of the gradient and along the flow.

### Example 11

### Statistical Analysis

Results from both in vitro and in vivo experiments were analyzed by Student's t test with p values≦50.05 taken as statistically significant.

### Example 12

### Single-Cell, Real-Time Responses Demonstrate the Direct Impact of RvD1 but not its Precursors DHA

The original isolation and structure elucidation of RvD1 demonstrated its presence and potent anti-inflammatory and pro-resolution actions in murine exudates and disease models in vivo^{23, 24}. The inventors questioned whether RvD1 or its precursor DHA has direct actions with human PMN and specifically whether RvD1 stops the directed movements of single cells along chemotactic gradients. To address this, a 1 μl microfluidic chemotaxis device that was engineered with microstructure membranes (see, EXAMPLE, 9 and FIGS. 8A-C) was used. The device permitted the isolation of single PMN from one drop of whole blood (see Methods and FIG. 8C) following a simple finger prick. This micro fluidic chamber was equipped with microscale valves pictured in FIG. 8B that were assembled to establish chemotactic gradients, with the chamber containing isolated single PMN that were captured from peripheral blood via finger stick venipuncture. These PMN and microfluidic chemotaxis device were recorded and continuously monitored using a CCD camera. As captured in FIG. 9A, these images show the abrupt cessation of chemotactic motility in response to RvD1.

A chemotactic gradient with the chemokine IL-8 was established in one of the chambers gradient networks, and human PMN were introduced into this chamber via the device inlet (illustrated in FIG. 6A). The PMN trafficked along with the IL-8 gradient (see, FIGS. 9B, 9C and 10). PMN exposed to the IL-8 gradient displayed the typical shape change and morphology of PMN during chemotaxis in a linear gradient (of ref.⁴⁰, FIG. 9A, left panel). Between 0 and 8 min, RvD1 at 10 nM was uniformly infused to the chamber. Before exposure to RvD1, individual PMN movements and distances were proportional to time and totaled ˜30-40 μm. Almost immediately on exposure to RvD1, PMN clearly changed shape (see FIG. 9A middle panel) and ceased directed chemotactic movements (FIGS. 9A and B). These changes were also captured on video (Videos 1 and 2, data not shown). FIG. 9B shows the average displacement, demonstrating a highly reproducible “breaking” or stopping of PMN migration when exposed to RvD1 (n=12). In sharp contrast, DHA, the biosynthetic precursor to RvD1, did not stop PMN migration at an equimolar dose (FIG. 10). Thus, by tracing single cells in the direction of an IL-8 chemotactic gradient and their displacement with RvD1 when introduced into the chamber, it was possible to record the direct actions of RvD1 with PMN and its ability to essentially completely stop PMN movements almost immediately upon exposure as well as RvD1's ability to stimulate rapid shape changes of PMN. These single cell recorded PMN responses were not shared by DHA (the RvD1 metabolic precursor) when introduced at equimolar concentrations in these microfluidic chemotaxis device.

### Example 13

### Comparison of Resolvin Analogs

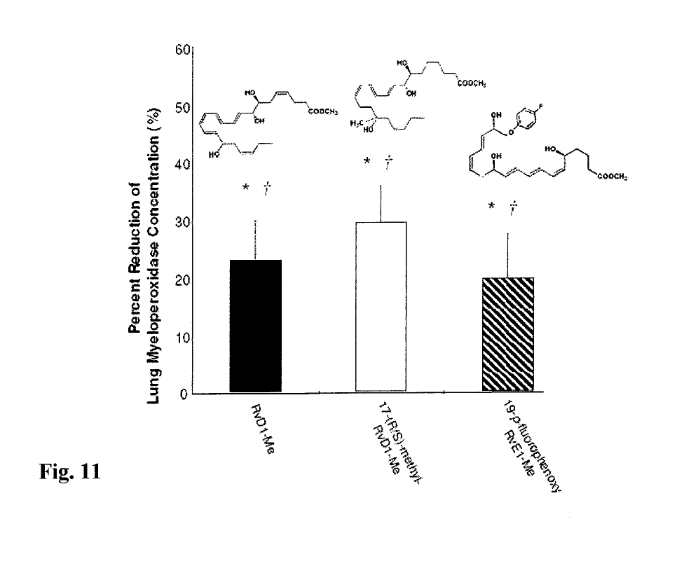

Using this system, the inventors compared the actions of RvD1 to its biosynthetic precursor DHA as well as to another resolvin, namely, RvE1 which is a potent product of EPA that is anti-inflammatory in both oral inflammation⁴³ and colitis.¹ RvD1, but neither RvE1 nor DHA, was able to protect the lung tissues from excessive leukocyte infiltration (FIG. 7C). The histology and reduction in leukocyte infiltration was confirmed by the reduction in the lung associated MPO values. RvD1 at 1 μg/mouse sharply reduced ˜50% leukocyte infiltration in the ischemia/reperfusion injury. At equal doses, neither DHA nor native RvE1 gave significant reduction in PMN tissue infiltration. Since RvD1 undergoes local metabolic inactivation²⁴ as does RvE1⁴⁴, blocking of their respective metabolic sites of inactivation de novo was undertaken using stable analog mimetics. To this end, both 17-(RIS)-methyl RvD1 and RvD1 methyl ester were prepared by total organic synthesis for in vivo administration. Both RvD1 and the related analogs reduced MPO levels in lung tissues (structures shown in FIG. 4A-C). Of interest, the metabolically stable analog of RvE1 namely, 1 9-p-fluorophenoxy RvE1 at 1 μg/mouse, also significantly reduced leukocyte infiltration while native RvE1 was inactive in this system. It is noteworthy that mouse lung tissue enzymatically converts RvE1 to 18-oxo-RvE1³⁰, which is devoid of activity. The 19-p-fluorophenoxy RvE1 prevents this metabolic inactivation (FIG. 11) and as documented here displayed potent protective actions dramatically reducing second organ injury of the lung.

### Example 14

### RvE1 Induces Bone Regeneration

The inventors established a small animal model for the evaluation of disease progression and regeneration after treatment using the rabbit as a model induced with periodontitis using Porphyromonas gingivalis. Briefly, ligatures were tied around the second mandibular premolar of rabbits for 6wk to create an environment where the periodontopatogen P. gingivalis could be retained. As negative controls, a group of animals only received ligatures without microbial challenge, another group did not receive an application and systemic metronidazole was used as a positive control to prevent P. gingivalis-induced infection. FIGS. 12A-12C show that RvE1 greatly limited the pathology of the induced gingivitis, not only with the soft tissue but also with the bone. In these figures the left panel shows the effect of the treatment on the soft tissue. The right panel is the same preparation but with the jaws defleshed to show the bone underneath. FIG. 12C shows that RvE1 treatment results in a restoration of normal architecture and regrowth of alveolar bone surrounding the teeth. FIG. 13 sows that the bone loss extends from the crest of the bone to the depth of the infrabony pocket. As illustrated the extent of the disease induced in marked (approximately 80%) and the RvE1-induced regeneration is to pre-disease levels (p<0.001).

### Example 15

### RvE1 Induces Bone Regeneration

In order to distinguish between new bone growth and true regeneration (reestablishment of the periodontal organ, undecalcified histologic sections of regenerated tissues were prepared. FIGS. 14A-C shows the establishment of new cementum, new fiber attachment and new bone and connective tissue in the area of prior periodontal disease. Placebo-treated animals exhibited no new bone or reattachment and are not shown. FIG. 14A shows the undecalcified ground section of the regenerated rabbit periodontium. FIG. 14B shows the phase contrast microscope image. FIG. 14C is the polarized light microscopic image. The figures illustrate the new deposition of the cementum (NC) the new periodontal ligament (PL), connective tissue (CT) and bone (B).

### Example 16

### RvE1 Inhibits Osteoclast Differentiation

In order to investigate the etiology of resolvins in bone regeneration, the inventors investigated the effect of RvE1 on osteoclast differentiation. In these experiments, peripheral blood monocytes were induced to differentiate into osteoclasts with RANKL treatment. Addition of either platelet rich plasma (PRP) known for its tissue saving effects and RvE1 significantly inhibited seacoast differentiation. FIG. 15 illustrates that the resorption area is significantly decreased in the presence of increases amounts of PRP or RvE1.

### CONCLUSION

Here, the direct actions of RvD1 on cell migration using human PMN as a model and was recorded in real time with the disclosed microvolume chemotaxis device.^{36, 60} As discussed herein, the microvolume chemotaxis device permitted monitoring of individual cell movements from a single drop of blood. This system permitted evaluating the activity of very small volumes of putative active members of the metabalome which included lipid mediators.

The techniques and methods discussed herein and afforded a unique opportunity to investigate the action of resolvins and protectins at the single cell level. The results from these in vitro experiments were then validated in vivo to identify previously unappreciated properties of the resolvin and protectin compound recited above as compounds I through LXXXIV.

The inventors have shown that circulating EPA and DHA, the biosynthetic precursors of lipid mediators, rapidly appeared in their unesterified forms at sites of inflammation where they can be utilized during resolution of inflammation for production of resolvins and protectins. Significantly the inventors further showed that these compounds previously appreciated for their anti-inflammatory activities at the site of inflammation, further acted to inhibit mobilization of migratory cells and, in particular, inflammatory cells, and their consequent migration to distant tissues and organs which would become the site of second organ injury following ischemia-reperfusion. These protective effects find great utility both therapeutically and prophylactically in many routine critical care instances such as transplant surgery, bypass surgery, and septic shock, for example. In addition, use of such second organ rescue actions include the addition of the resolvins and protectins of the invention added to the milieu of the transplant organ prior following the explant and prior to the transplant in order to prevent tissue damage locally.

Further, the results provided herein demonstrate the unappreciated action of the resolvin and protectin compounds disclosed herein to protect and remediate damage to connective tissue and connective tissue degeneration. The investigations described above, show the protection of ligament and connective tissue but also illustrate their effectiveness in encouraging new growth and regeneration. Such conditions include but are not limited to arthritis, diabetes, gout, osteoarthritis, Lyme disease, Perthe's disease, mechanical injury, alkaptonuria, or hemochromatosis or periodontal disease.

In addition, the investigations illustrate that the resolvin and protectin compounds described not only reduce the effect of disease on bone loss but significantly encourage new growth and provide restorative actions. These effects should be widely useful in treating such debilitating diseases such as osteoporosis and in treating the effects of bone loss due to infection and the immune response such as, for example osteoarthritis, periodontitis and the like

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this invention, as set forth above, are intended to be illustrative not limiting. Various changes may be made without departing from the spirit and scope of the invention. therefor3e, the invention is intended to embrace all known or later-developed alternatives, modifications, variations, improvements and/or substantial equivalents of these exemplary embodiments.

### CITED REFERENCES

1. Serhan, C. N. Resolution phases of inflammation: novel endogenous anti-inflammatory and pro-resolving lipid mediators and pathways. Annu. Rev. Immunol. 25, 101-137 (2007).
2. Majno, G. & Joris, I. Cells, Tissues, and Disease: Principles of General Pathology, Edn. 2nd. (Oxford University Press, New York; 2004).
3. Weissmann, G. Pathogenesis of rheumatoid arthritis. J. Clin. Rheumatol. 10(3 Suppl), S26-S31 (2004).
4. Kantarci, A. & Van Dyke, T. E. Resolution of inflammation in periodontitis. J. Periodontol. 76, 2168-2174 (2005).
5. Nathan, C. Points of control in inflammation. Nature 420, 846-852 (2002).
6. Libby, P. Inflammation in atherosclerosis. Nature 420, 868-878 (2002).
7. Bazan, N. G., Colangelo, V. & Lukiw, W. J. Prostaglandins and other lipid mediators in Alzheimer's disease. Prostaglandins Other Lipid Mediat. 68-69, 197-210 (2002).
8. Gilroy, D. W., Lawrence, T., Perretti, M. & Rossi, A. G. Inflammatory resolution: new opportunities for drug discovery. Nat. Rev. Drug Discov. 3, 401-416 (2004).
9. Schwab, J. M., Chiang, N., Arita, M. & Serhan, C. N. Resolvin E1 and protectin D1 activate inflammation-resolution programmes. Nature 447, 869-874 (2007).
10. Maddox, J. F. & Serhan, C. N. Lipoxin A₄ and B₄ are potent stimuli for human monocyte migration and adhesion: selective inactivation by dehydrogenation and reduction. J. Exp. Med. 183, 137-146 (1996).
11. Reville, K., Crean, J. K., Vivers, S., Dransfield, I. & Godson, C. Lipoxin A4 redistributes myosin IIA and Cdc42 in macrophages: implications for phagocytosis of apoptotic leukocytes. J. Immunol. 176, 1878-1888 (2006).
12. Godson, C. et al. Cutting edge: Lipoxins rapidly stimulate nonphlogistic phagocytosis of apoptotic neutrophils by monocyte-derived macrophages. J. Immunol. 164, 1663-1667 (2000).
13. Canny, G. et al. Lipid mediator-induced expression of bactericidal/permeability-increasing protein (BPI) in human mucosal epithelia. Proc. Natl. Acad. Sci. USA 99, 3902-3907 (2002).
14. GISSI-Prevenzione Investigators Dietary supplementation with n-3 polyunsaturated fatty acids and vitamin E after myocardial infarction: results of the GISSI-Prevenzione trial. Gruppo Italiano per lo Studio della Sopravvivenza nell'Infarto miocardico. Lancet 354, 447-455 (1999).
15. Marchioli, R. et al. Early protection against sudden death by n-3 polyunsaturated fatty acids after myocardial infarction: time-course analysis of the results of the Gruppo Italiano per lo Studio della Sopravvivenza nell'Infarto Miocardico (GISSI)-Prevenzione. Circulation 105, 1897-1903 (2002).
16. Leeb, B. F., Sautner, J., Andel, I. & Rintelen, B. Intravenous application of omega-3 fatty acids in patients with active rheumatoid arthritis. The ORA-1 trial. An open pilot study. Lipids 41, 29-34 (2006).
17. Pluess, T. T. et al. Intravenous fish oil blunts the physiological response to endotoxin in health subjects. Intensive Care Med. 33, 789-797 (2007).
18. Welty, F. K., Lee, K. S., Lew, N. S. & Zhou, J. R. Effect of soy nuts on blood pressure and lipid levels in hypertensive, prehypertensive, and normotensive postmenopausal women. Arch. Intern. Med. 167, 1060-1067 (2007).
19. Norris, J. M. et al. Omega-3 polyunsaturated fatty acid intake and islet autoimmunity in children at increased risk for type 1 diabetes. JAMA. 298, 1420-1428 (2007).
20. Lefkowitz, W., Lim, S. Y., Lin, Y. & Salem, N. N., Jr. Where does the developing brain obtain its docosahexaenoic acid: relative contributions of dietary alpha-linoleic acid, docosahexaenoic acid, and body stores in the developing rat. Pediatr. Res. 57, 157-165 (2005).
21. Bazan, N. G. Omega-3 fatty acids, pro-inflammatory signaling and neuroprotection. Curr. Opin. Nutr. Metab. Care 10, 136-141 (2007).
22. Freedman, S. D. et al. Association of cystic fibrosis with abnormalities in fatty acid metabolism. N. Engl. J. Med. 350, 560-569 (2004).
23. Serhan, C. N. et al. Resolvins: a family of bioactive products of omega-3 fatty acid transformation circuits initiated by aspirin treatment that counter pro-inflammation signals. J. Exp. Med. 196, 1025-1037 (2002).
24. Sun, Y.-P. et al. Resolvin D1 and its aspirin-triggered 17R epimer: stereochemical assignments, anti-inflammatory properties and enzymatic inactivation. J. Biol. Chem. 282, 9323-9334 (2007).
25. Gong, J. et al. Plasma docosahexaenoic acid levels in various genetic forms of retinitis pigmentosa. Invest. Ophthalmol. Vis. Sci. 33, 2596-2602 (1992).
26. Newcomer, L. M., King, I. B., Wicklund, K. G. & Stanford, J. L. The association of fatty acids with prostate cancer risk. Prostate 47, 262-268 (2001).
27. Albert, C. M. et al. Blood levels of long-chain n-3 fatty acids and the risk of sudden death. N. Engl. J. Med. 346, 1113-1118 (2002).
28. Kew, S. et al. Effects of oils rich in eicosapentaenoic and docosahexaenoic acids on immune cell composition and function in healthy humans. Am. J. Clin. Nutr. 79, 674-681 (2004).
29. Wakai, K. et al. Intake frequency of fish and serum levels of long-chain n-3 fatty acids: a cross-sectional study within the Japan Collaborative Cohort Study. J. Epidemiol. 15, 211-218 (2005).
30. Arita, M. et al. Metabolic inactivation of resolvin E1 and stabilization of its anti-inflammatory actions. J. Biol. Chem. 281, 22847-22854 (2006).
31. Arita, M. et al. Stereochemical assignment, anti-inflammatory properties, and receptor for the omega-3 lipid mediator resolvin E1. J. Exp. Med. 201, 713-722 (2005).
32. Bradford, M. M. A rapid and sensitive method for the quantitation of microgram quantities of protein utilizing the principle of protein-dye binding. Anal. Biochem. 72, 248-254 (1976).
33. Ariel, A. et al. Apoptotic neutrophils and T cells sequester chemokines during immune response resolution via modulation of CCR5 expression. Nat. Immunol. 7, 1209-1216 (2006).
34. deBoer, T. J. & Backer, H. J. A new method for the preparation of diazomethane. Recl. Tray. Chim. Pays. Bas. 73, 229-234 (1954).
35. Qiu, F.-H., Wada, K., Stahl, G. L. & Serhan, C. N. IMP and AMP deaminase in reperfusion injury down-regulates neutrophil recruitment. Proc. Natl. Acad. Sci. USA 97, 4267-4272 (2000).

36. Irimia, D. & Toner, M. Cell handling using microstructured membranes. Lab Chip 6, 345-352 (2006).
37. Winyard, P. G. & Willoughby, D. A. (eds.) Inflammation Protocols. (Humana, Totowa, N.J.; 2003).
38. Meyers, D. G., Meyers, R. E. & Prendergast, T. W. The usefulness of diagnostic tests on pericardial fluid. Chest 111, 1213-1221 (1997).
39. Bannenberg, G. L. et al. Molecular circuits of resolution: Formation and actions of resolvins and protectins. J. Immunol. 174, 4345-4355 (2005).
40. Geiser, T., Dewald, B., Ehrengruber, M. U., Clark-Lewis, I. & Baggiolini, M. The interleukin-8-related chemotactic cytokines CROalpha, GRObeta, and GROgamma activate human neutrophil and basophil leukocytes. J. Biol. Chem. 268, 15419-15424 (1993).
41. Cotran, R. S., Kumar, V. & Collins, T. (eds.) Robbins Pathologic Basis of Disease, Edn. 6th. (W.B. Saunders Co., Philadelphia; 1999).
42. Gelman, S. The pathophysiology of aortic cross-clamping and unclamping. Anesthesiology 82, 1026-1060 (1995).
43. Hasturk, H. et al. Resolvin El regulates inflammation at the cellular and tissue level and restores tissue homeostasis in vivo. J. Immunol. 179, 7021-7029 (2007).
44. Hong, S. et al. Resolvin E1 metabolome in local inactivation during inflammation-resolution. J. Immunol. 180, 3512-3519 (2008).
45. Arterburn, L. M., Hall, E. B. & Oken, H. Distribution, interconversion, and dose response of n-3 fatty acids in humans. Am. J. Clin. Nutr. 83, 1467S-1476S (2006).
46. Hong, S., Gronert, K., Devchand, P., Moussignac, R.-L. & Serhan, C. N. Novel docosatrienes and 175-resolvins generated from docosahexaenoic acid in murine brain, human blood and glial cells: autacoids in anti-inflammation. J. Biol. Chem. 278, 14677-14687 (2003).
47. Marcheselli, V. L. et al. Novel docosanoids inhibit brain ischemia-reperfusion-mediated leukocyte infiltration and pro-inflammatory gene expression. J. Biol. Chem. 278, 43807-43817 (2003).
48. Serhan, C. N. & Chiang, N. Endogenous pro-resolving and anti-inflammatory lipid mediators: a new pharmacologic genus. Br. J. Pharmacol. 153, S200-S215 (2008).
49. Patil, V. S. & Magar, N. G. Fatty acids of human blood. Biochem. 1 74, 427-429 (1960).
50. Burdge, G. C. & Calder, P. C. Conversion of alpha-linolenic acid to longer-chain polyunsaturated fatty acids in human adults. Reprod. Nutr. Dev. 45, 581-597 (2005).
51. Pawlosky, R. J., Hibbeln, J. R., Novotny, J. A. & Salem, N. N., Jr. Physiological compartmental analysis of alpha-linolenic acid metabolism in adult humans. J. Lipid Res. 42, 1257-1265 (2001).
52. U.S. Food and Drug Administration Center for Food Safety and Applied Nutrition Office of Nutritional Products Labeling and Dietary Supplements (FDA/CSFAN). Letter regarding dietary supplement health claim for omega-3 fatty acids and coronary heart disease (Docket No. 91N-0103). (2000).
53. Lands, B. A critique of paradoxes in current advice on dietary lipids. Prog. Lipid Res. 47, 77-106 (2008).
54. Samuelsson, B., Dahlen, S. E., Lindgren, J. A., Rouzer, C. A. & Serhan, C. N. Leukotrienes and lipoxins: structures, biosynthesis, and biological effects. Science 237, 1171-1176 (1987).
55. Huang, B. X., Dass, C. & Kim, H. Y. Probing conformational changes of human serum albumin due to unsaturated fatty acid binding by chemical cross-linking and mass spectrometry. Biochem. J. 387, 695-702 (2005).
56. Böyum, A. Isolation of leucocytes from human blood. Further observations. Methylcellulose, detran, and ficoll as erythrocyteaggregating agents. Scand. J. Clin. Lab. Invest. Suppl. 97, 31-50 (1968).
57. Boyden, S. The chemoactic effect of mixtures of antibody and antigen on polymorphonuclear leucocytes. J. Exp. Med. 115, 453-466 (1962).
58. Zicha, D., Dunn, G. A. & Brown, A. F. A new direct-viewing chemotaxis chamber. J. Cell Sci. 99, 769-775 (1991).
59. Zigmond, S. H. & Hirsch, J. G. Leukocyte locomotion and chemotaxis. New methods for evaluation, and demonstration of a cell-derived chemotactic factor. J. Exp. Med. 137, 387-410 (1973).
60. Irimia, D. et al. Microfluidic system for measuring neutrophil migratory responses to fast switches of chemical gradients. Lab Chip 6, 191-198 (2006).
61. Carillo-Rivas, B. et al. Resolvin E1 inhibits collagen-induced arthritis and pro-inflammatory cytokine production in rodents. 10th International Conference on Bioactive Lipids in Cancer, Inflammation and Related Diseases (Abstract #82) (2007).

What is claimed is:

1. A method for treating or preventing second organ injury resulting from ischemia-reperfusion comprising administering to a patient suffering or at risk of suffering second organ injury resulting from ischaemia-reperfusion comprising a therapeutically effective amount of a resolvin or protectin selected from compounds I through LXXXIV.

2. The method of claim 1, wherein the resolvin is administered prophylactically.

3. The method of claim 1, wherein the resolvin is administered therapeutically.

4. The method of claim 1, wherein the resolvin is administered orally, rectally topically, intravenously, intraperitoneally, as an inhalant, as a mist as a tablet, a capsule, a tincture, as an implantable matrix.

5. The method of claim 1, wherein the second organ injury results from transplant surgery, bypass surgery, septic shock, and explant surgery

6. A method of treating, preventing or ameliorating connective tissue degeneration in a patient in need thereof, comprising administering a therapeutic amount of a resolvin or protectin selected from compounds I through LXXXIV.

7. The method of claim 6, wherein the resolvin or protectin is administered orally, rectally topically, intravenously, intraperitoneally, as an inhalant, as a mist as a tablet, a capsule, a tincture, as an implantable matrix.

8. The method of claim 7 wherein the implantable matrix is a hydrogel or an osmotic pump.

9. The method of claim 6, wherein the connective tissue degeneration results from arthritis, diabetes, gout, Lyme disease, Perthe's disease, mechanical injury, alkaptonuria, hemochromatosis, osteoarthritis or periodontal disease.

10. A method of treating or preventing bone loss comprising administering a therapeutically effective amount of a resolvin or protectin selected from compounds I through LXXXIV.

11. The method of claim 10, wherein the bone loss results from osteoporosis, osteoarthritis or periodontal disease.

12. The method of claim 10, wherein the resolvin or protectin is administered orally, rectally topically, intravenously, intraperitoneally, as an inhalant, as a mist as a tablet, a capsule, a tincture, as an implantable matrix.

13. The method of claim 10, wherein the implantable matrix is a hydrogel or an osmotic pump.

14. The method of claim 10, wherein the treatment results in bone regeneration.
